(19) United States
(12) Patent Application Publication
(10) Pub. No.: US 2026/0311892 A1
(43) Pub. Date: Oct. 8, 2026

(54) HOSPITAL PATIENT COMMUNICATION SYSTEM FOR HALLWAY BEDS WITH INTEGRATED EMERGENCY ALERTS AND TWO-WAY VOICE COMMUNICATION

(71) Applicant: Vital Tech Medical Solutions, Inc., Newark, DE (US)

(72) Inventor: Olincia White, Sacramento, CA (US)

(73) Assignee: VITAL TECH MEDICAL SOLUTIONS, INC., Newark, DE (US)

(21) Appl. No.: 19/438,614

(22) Filed: Jan. 1, 2026

Related U.S. Application Data
(63) Continuation-in-part of application No. 19/172,660, filed on Apr. 8, 2025.

(51) Int. Cl. G16H 80/00; G16H 40/20; G16H 40/67

(52) U.S. Cl. CPC G16H 80/00; G16H 40/20; G16H 40/67

(57) ABSTRACT

A wireless call light device for use in healthcare environments is disclosed. The device includes a communication portal configured to transmit patient-initiated requests to a receiving system, a speaker and microphone enabling two-way audio communication, and a processor and memory storing a user-programmable identifier corresponding to a patient support apparatus. An indicator light illuminates upon call initiation and remains active until reset via an authorized reset mechanism, while a latched call state suppresses repeated inputs. Patient input may be received via a physical control or via an artificial intelligence interface executed by the processor. The artificial intelligence interface interprets patient input and determines device actions, including generation of standardized assistance events. The device may include multimodal output, a hot-swappable power source, and mounting mechanisms for attachment to patient support apparatuses or IV poles. Artificial intelligence processing may be performed locally, remotely, or in a hybrid configuration.

### CROSS-REFERENCE TO RELATED APPLICATIONS

This application is a continuation-in-part of U.S. application Ser. No. 19/172,660, filed Apr. 8, 2025, the entirety of which is incorporated herein by reference.

### FIELD OF THE INVENTION

The present invention relates to healthcare communication systems, specifically a wireless communication device designed to enhance emergency and non-emergency communication between patients and healthcare staff in hospital settings. More particularly, the invention provides a system that allows patients, particularly those located in hallway beds or other non-traditional locations, to easily communicate with healthcare personnel through a portable, wireless device with integrated visual and audio features. The system improves the response time for medical assistance, increases patient safety, and streamlines the workflow of healthcare staff.

### BACKGROUND OF THE INVENTION

Healthcare facilities commonly employ nurse call or call light systems to enable patients to request assistance from clinical staff. Traditional call light systems are typically hardwired to fixed infrastructure within patient rooms and are designed for use in permanent bed locations. These systems generally rely on a physical call button connected to a wall-mounted control unit or centralized nurse station, and often lack flexibility for temporary, mobile, or non-room patient placements.

Modern healthcare environments increasingly experience conditions in which patients are placed outside of traditional patient rooms, such as in hallways, overflow areas, triage spaces, or temporary care locations, particularly during periods of high patient volume or emergency conditions. In such environments, fixed nurse call infrastructure may be unavailable, inaccessible, or impractical to deploy, leaving patients without a reliable means to request assistance or communicate with staff.

Existing portable or wireless call light solutions often provide limited functionality and may lack reliable location identification, secure reset mechanisms, or persistent call indication. Many such systems are susceptible to nuisance resets, repeated call activations, or ambiguous signaling that can reduce staff confidence and increase response delays.

Additionally, conventional call light devices typically require manual button actuation and may be inaccessible to patients with limited mobility, speech impairments, or reduced motor control.

Further, traditional call light systems are generally not configured to interpret non-button patient input, such as spoken requests, non-verbal vocalizations, or unclear speech patterns, and therefore may fail to capture or prioritize patient needs when patients are unable to actuate a physical control. When voice-enabled systems are used, they often lack reliable mechanisms for assessing interpretation confidence, handling ambiguous input, or providing fallback procedures to ensure patient safety.

Existing systems also commonly lack integrated mechanisms for persistent call state management, such as latched call states that prevent repeated activations until a request is acknowledged and cleared by authorized staff. Inadequate reset controls may allow patients or unauthorized users to prematurely clear calls, leading to missed assistance requests. Conversely, overly restrictive systems may require physical presence at a fixed location to clear calls, reducing workflow efficiency.

Additionally, many current nurse call and communication systems do not provide structured, event-based signaling suitable for integration with modern hospital information systems, mobile staff devices, or distributed communication architectures. The absence of standardized event representations and acknowledgment handling can result in unreliable delivery, lack of confirmation, and poor interoperability across devices and systems.

Accordingly, there is a need for improved call light devices and systems that are portable, wireless, and configurable for use in both traditional patient rooms and non-room environments such as hallways. There is a further need for devices capable of securely associating with patient support apparatuses, providing persistent and reliable call indication, enabling two-way communication, and supporting both physical and voice-based patient input.

There is also a need for systems that can intelligently interpret patient input, assess confidence, generate standardized assistance events, and implement safe fallback behavior when interpretation is uncertain, while maintaining secure reset control and reliable communication with receiving systems.

In addition, healthcare delivery environments continue to evolve toward more flexible, technology-enabled workflows that emphasize patient accessibility, staff efficiency, and system interoperability. Devices that support adaptable deployment, intuitive patient interaction, and seamless integration with existing clinical infrastructure are increasingly desirable. Solutions that combine portable hardware, secure communication, and software-driven control logic can improve responsiveness, reduce workflow friction, and enhance patient experience without requiring extensive physical installation or modification of facility infrastructure. Accordingly, systems that provide configurable, resilient, and patient-centric communication capabilities are well suited to address the operational demands of modern healthcare settings.

### SUMMARY OF THE INVENTION

The present invention relates to a wireless communication system specifically designed for use in hospital environments where patients might be in temporary beds, hallway beds, or emergency room (ER) situations. This system aims to address the communication challenges between patients and medical staff, particularly when patients do not have easy access to traditional call systems found in designated patient rooms.

The invention provides an integrated communication tool that is not only wireless and portable but also simple to operate, ensuring immediate and efficient communication with medical staff during emergencies or routine requests. By incorporating, but not limited to a communication module with a button-activated call system, two-way speakerphone communication, a visual indicator light, and specialized emergency buttons, the invention ensures that patients in overflow or hallway beds can easily request assistance, with clear alerts sent to the appropriate medical staff for timely response.

This invention is particularly valuable in emergency settings where quick access to medical personnel can mean the difference between life and death. Additionally, it enhances hospital workflow, increases operational efficiency, and improves overall patient experience.

The heart of the system is the communication module, which enables the device to place calls and transmit signals wirelessly. This module is designed to connect to the hospital's central communication infrastructure (e.g., nurse call systems, electronic health records). When activated, the communication module transmits a signal to alert medical personnel, ensuring the patient's request is logged and the staff is aware of the situation.

The communication module can use Wi-Fi, Bluetooth, or RF communication protocols, making it flexible in terms of hospital network architecture. Its integration with hospital databases means it can trigger alerts, update patient status, and inform staff about ongoing requests, all in real-time.

The primary method by which the patient initiates communication is via the call button. This button can be placed directly on the patient's bed or attached to a flexible cord that the patient can hold or clip onto their clothing. Once pressed, the button sends a wireless signal to the communication module, indicating that the patient requires assistance.

The design of the call button is intuitive, ensuring ease of use for patients with varying levels of mobility or cognitive ability. The button is large enough to be pressed easily by patients in a variety of positions, including those who may be bedridden, elderly, or physically challenged. The device can also accommodate multiple buttons-including the standard call button, along with specialized buttons for emergency situations like Code Blue and Rapid Response.

The device is equipped with a speakerphone system to enable two-way communication between the patient and medical staff. This feature is essential for situations where the patient cannot physically interact with the device but needs to explain their condition. The speakerphone allows for a real-time conversation between the patient and hospital staff, allowing the latter to assess the urgency of the situation, offer instructions, or prioritize medical intervention.

The microphone and speaker system are designed to be high-quality, ensuring clear communication even in noisy hospital environments. The microphone is sensitive enough to pickup the patient's voice clearly, while the speaker is loud enough to be heard over ambient hospital noise. This feature improves the patient's ability to communicate vital information, such as pain levels, symptoms, or medical history, directly to the attending medical staff.

An innovative feature of the invention is the indicator light. When a call is initiated, a visual alert is activated. The light remains on until it is manually turned off by a member of the medical team, indicating that the request for assistance has been received and is being processed. This persistent visual indicator makes it easier for hospital staff to see if any patient requires attention, even if they are not in the immediate vicinity.

This indicator light provides an extra level of security, especially in high-traffic hospital environments where traditional call systems might go unnoticed or where staff cannot always hear auditory signals. The light can be placed on a variety of devices and is designed to remain visible from a distance, ensuring that staff are alerted to a patient's request no matter where they are in the hospital.

The device is equipped with dedicated emergency buttons designed for urgent situations like Code Blue (cardiac or respiratory arrest) or Rapid Response (for patients exhibiting deteriorating conditions). These buttons are easy to access and clearly marked, allowing patients to quickly signal life-threatening situations.

Pressing one of these emergency buttons sends an immediate alert to hospital staff with a higher priority than the regular call button, ensuring that critical situations are escalated appropriately. These emergency signals trigger special visual and audio alerts in the central hospital system, ensuring that medical teams are mobilized without delay.

The device operates on a rechargeable battery housed in a battery compartment. The power source is designed for long-term use, providing sufficient charge for extended periods without frequent recharging. This is particularly important in emergency room environments, where the device must function continuously, even during long shifts or busy hospital hours.

Additionally, the device is equipped with a low-battery warning system to alert medical staff when the battery is running low. This ensures that the device is always operational and ready for use when needed.

The device is designed to be portable, allowing it to be used in a variety of hospital settings. It can be mounted on a hospital bed, clipped onto a gurney, or placed on medical equipment such as IV poles or monitoring systems. This versatility ensures that patients can use the device in overflow beds, temporary beds, or emergency treatment areas where traditional call systems may not be available.

The mounting options make the device easy to use without requiring additional setup, reducing the burden on hospital staff and ensuring seamless integration into existing healthcare infrastructure. The design is intended to ensure that patients in any hospital location can use the device without confusion or difficulty.

One of the most powerful features of this invention is the centralized communication portal. This portal is integrated with the hospital's existing communication systems, allowing staff to manage all incoming calls, prioritize them based on urgency, and track responses in real time.

The portal enables the hospital to maintain an accurate record of patient calls and responses, helping to optimize resource allocation and improve patient care coordination. Staff can access the portal from any device, including smartphones, tablets, or hospital workstations, making it easy to respond to requests no matter where they are located.

The invention is designed to seamlessly integrate with a hospital's existing electronic health records (EHR) and nurse call systems. This means that when a call is made, it not only triggers an alert in the central system but also logs the request in the patient's medical record. This integration ensures that staff have access to relevant patient information in real time, allowing them to make informed decisions quickly.

Additionally, the integration with nurse call systems allows hospital staff to prioritize calls based on the patient's condition, ensuring that those requiring urgent medical intervention receive timely attention. The device is thus a vital tool in enhancing the efficiency and effectiveness of hospital operations, improving both patient care and the hospital's ability to manage high patient volumes.

Several other features can be incorporated into the device, depending on hospital needs. For example, the device can include a display screen to show important information such as the patient's name, the status of the call, or a brief description of the medical condition. This allows staff to quickly assess the situation before responding.

The system can also be upgraded with additional sensors, but not limited to, such as motion detectors or vital sign monitors, that provide real-time data to medical staff. For example, if a patient in distress presses the emergency button, the system could automatically send alerts that include the patient's heart rate, blood pressure, or oxygen saturation, enabling medical staff to take swift action.

### Advantages of the Invention

The wireless communication tool significantly improves patient safety by providing a reliable and efficient means for patients to request help. The persistent indicator light ensures that medical staff are always aware of the patient's request, even when they are not within earshot. This leads to faster responses and fewer missed calls.

By integrating with existing hospital systems, the device streamlines workflow by ensuring that requests are automatically logged, prioritized, and tracked. This integration helps hospitals manage patient care more effectively, reducing the risk of errors and delays.

The wireless communication tool is highly scalable and can be deployed across hospitals of any size. Its portability ensures that it can be used in overflow areas, while its ability to integrate with existing systems makes it a cost-effective solution for improving hospital communication.

The ability to make an instant connection with the medical staff and provide real-time information reduces response time, leading to improved patient outcomes. The two-way communication system allows staff to assess the situation remotely, potentially taking action without needing to be physically present.

This wireless communication tool offers a comprehensive solution to the problem of communication breakdowns in hospital settings, particularly for patients in temporary beds or hallway beds. Its ability to improve patient safety, streamline hospital workflows, and enhance emergency response makes it an essential device for modern healthcare environments. The system's flexibility, integration capabilities, and ease of use ensure that it can meet the needs of hospitals, whether in large urban centers or smaller community-based facilities. By providing a portable and reliable communication tool, the invention has the potential to revolutionize how hospitals manage patient care, particularly in times of high demand and critical need.

### BRIEF DESCRIPTION OF THE ACCOMPANYING DRAWINGS

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a perspective view of the wireless call light device (100);

FIG. 2 is a front elevation view of the wireless call light device (100) showing the indicator light (102) and patient call button (106);

FIG. 3 is a rear elevation view of the wireless call light device (100) showing the staff-accessible buttons;

FIG. 4 is a side elevation view of the wireless call light device (100) illustrating the grip (118) and battery compartment (120);

FIG. 5 is a block diagram of the primary components of the wireless call light device (100) are portable electronic device (122), including memory (124), processor (126), communication portal (128), and power source (130);

FIG. 6 is a detailed view of the device (100) attached to an IV pole using the holding clip (132) and showing the rod (134);

FIG. 7 is a close-up view of the patient-accessible buttons, specifically the patient call button (106) and speaker (108);

FIG. 8A and FIG. 8B are a close-up view of the staff-accessible buttons on the rear of the device and patient-accessible buttons;

FIG. 9 is illustrating the diagrams of wireless call light device (100), which mechanically coupled to a patient bed in accordance with exemplary embodiments of the present invention;

FIG. 10 is a block diagram illustrating a wireless call light devices communication with the central communication module according to the present invention;

FIG. 11 is a detailed block diagram of the device's electronic components, including the microphone (144), audio interface (146), speaker (108), radio (148), and DSP (150);

FIG. 12 is a flowchart illustrating the device's operational sequence;

FIG. 13 is a flowchart illustrate the sequence of steps involved when a patient presses the call button on the device, the communication that takes place between the patient and the healthcare staff, and the resulting actions; and

FIG. 14 is a flowchart describe the steps involved when staff members interact with the device for critical scenarios such as a Code Blue or Rapid Response situation.

### DETAILED DESCRIPTION OF THE INVENTION

The invention addresses a significant gap in the existing communication infrastructure within hospital emergency departments (ED) and similar healthcare settings. Specifically, it focuses on the wireless communication system that enables patients in hallway beds or non-traditional locations to have direct, real-time communication with medical staff. The system comprises a patient call button, a central communication portal, and several features designed to ensure timely assistance in emergency situations, including visual indicators, audio interfaces, and manual override functions.

The device is designed to improve the workflow of the medical staff, enhance patient safety, and ensure efficient communication in situations where traditional call light systems may not be adequate, particularly in hallway settings.

The wireless communication system consists of several key components that work together seamlessly to ensure reliable and effective communication between the patient and the hospital staff.

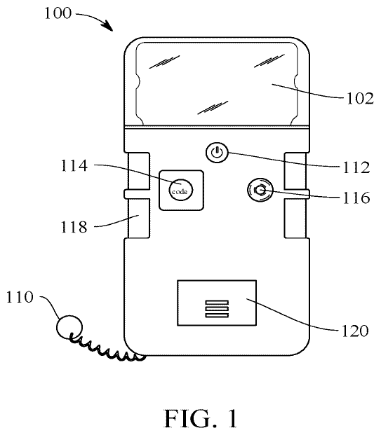

FIG. 1 illustrates an embodiment of the wireless call light device (100) in a perspective view. The device includes an enclosure (104) that houses the internal components and provides structural integrity. The enclosure (104) includes an indicator light (102) is positioned on the front face of the device, serving as a visual alert when the patient initiates a call. Further, the enclosure (104) also includes a patient call button (106) is mounted on the device, allowing patients to request assistance. The speaker (108) is integrated into the enclosure to enable two-way audio communication between the patient and hospital staff. This embodiment provides an overall view of the major external elements of the device.

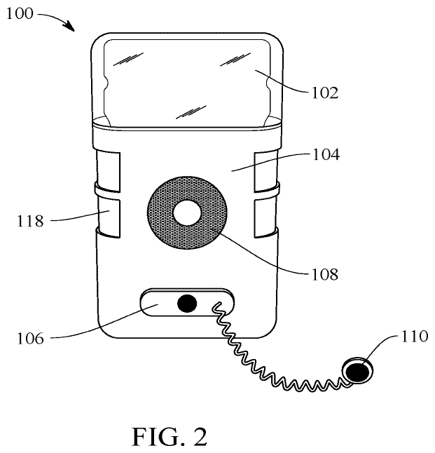

FIG. 2 presents a front view of the device (100), showing key patient-accessible features. The indicator light (102) is positioned prominently to provide a clear visual signal when activated. The patient call button (106) is placed ergonomically to be easily accessible for patients. The speaker (108) is located in the enclosure to facilitate audio interaction between the patient and medical personnel. This embodiment ensures an intuitive user interface, making it easy for patients to operate the device.

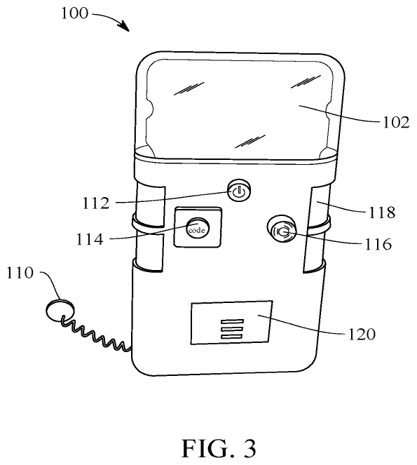

FIG. 3 illustrates the rear view of the device (100), where staff-accessible buttons are located. This embodiment includes a power button (112) to turn the device on and off. A code blue button (114) is incorporated for emergency situations requiring immediate intervention. Similarly, a rapid response button (116) is provided to enable urgent medical alerts. These buttons are positioned on the back of the device to prevent accidental activation by the patient while ensuring easy accessibility for medical personnel.

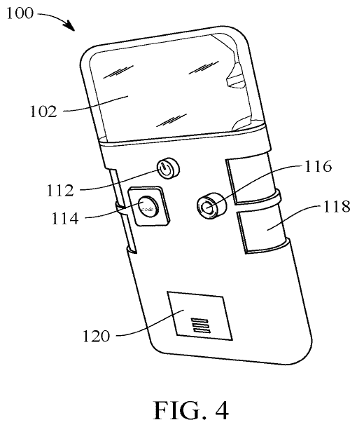

FIG. 4 depicts a side view of the device (100), highlighting the grip (118) and battery compartment (120). The grip (118) ensures secure handling, preventing accidental falls or displacements. The battery compartment (120) is designed for easy battery replacement and supports a hot-swappable battery (130) to ensure uninterrupted operation. This embodiment showcases the ergonomic design of the device, making it convenient for both patients and healthcare providers.

The indicator light (102) is a crucial element of the invention, serving both as a visual alert for the medical staff and as a confirmation of an active communication session. The light is activated whenever the patient call button (106) is pressed. The light remains illuminated until manually turned off by a staff member, ensuring that even if the clerk or nurse is otherwise occupied, another staff member passing by the patient can quickly identify the need for assistance. Additionally, the indicator light can have a color-coded system to signify the urgency of the request (e.g., yellow for normal, red for urgent, blue for Code Blue, and flashing red for Rapid Response).

The enclosure or case of the device (104) houses all the internal components of the system, providing protection and facilitating ease of use. It is designed to be robust, yet lightweight, with an ergonomic shape that allows it to be easily attached to a hospital bed or IV pole using a clamp. The enclosure also includes a battery compartment (120) to house the swappable battery, ensuring continuous operation throughout the day. It also houses the communication module, which is responsible for sending and receiving signals between the patient device and the hospital communication system.

The patient call button (106) is the primary mechanism for the patient to request assistance. This button is located on the device's body or mounted on a flexible cord (110), which is typically attached to a wand that the patient can easily hold. When the button is pressed, it immediately sends a signal to the hospital communication system to initiate the call. This button may also include a light to indicate that the signal has been successfully sent.

The speaker (108) is embedded within the device to allow two-way communication between the patient and the hospital staff. It functions alongside the microphone (144), creating a speakerphone-like feature that enables real-time communication. The patient can speak to the medical staff via the speaker, and the staff can respond to the patient through the same interface.

A Code Blue button (114) is included as an emergency feature that allows the patient or staff to signal a life-threatening emergency. When pressed, the light on the device will flash blue, and a signal will be sent to the hospital's central communication portal, indicating that immediate action is required. This functionality ensures that medical personnel can respond to critical situations as quickly as possible.

Similarly, the Rapid Response button (116) serves to indicate an urgent situation requiring immediate attention. When pressed, the device's light will flash red, and a signal will be sent to alert the hospital staff. This button is designed for less critical, but still urgent, medical situations that require swift intervention.

The grip (118) is incorporated into the design of the device to make it easier for staff to handle and move the device when required. It is typically located on the back or side of the unit and is ergonomically positioned to allow a comfortable and firm hold.

The device is powered by a hot-swappable battery stored in the battery compartment (120). The swappable feature allows the device to be easily recharged or replaced without requiring the entire system to be taken out of service. A charging base is included to allow the device to be placed for charging when not in use.

The wireless call light device (100) includes a portable electronic device (122) that refers to the entirety of the wireless communication system, designed to be mobile and easy to transport within a hospital environment. The device is lightweight, compact, and can be clipped to various locations near the patient, including IV poles, gurneys, or other patient-supporting equipment.

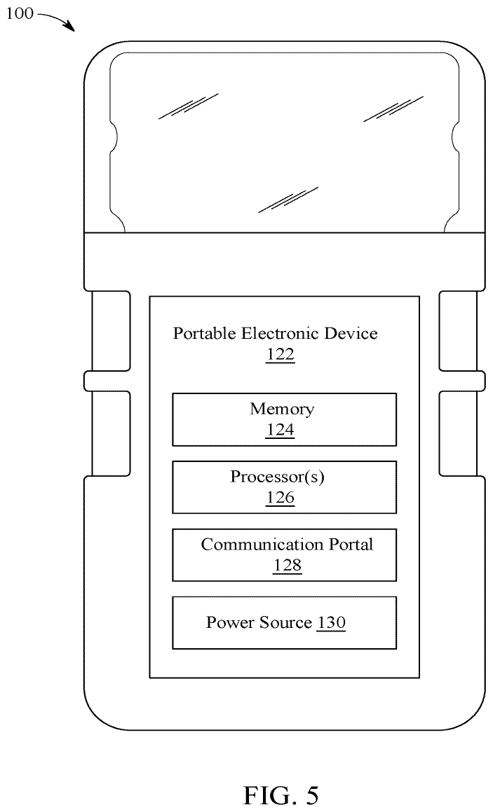

FIG. 5 illustrates a schematic representation of the internal components of the wireless call light device (100). The wireless call light device (100) includes a portable electronic device (122). Further, the portable electronic device (122) includes a memory (124), a processor (126), a communication portal (128) and power source (130). The memory (124) stores configuration settings and operational data. The processor (126) controls various functions of the device, including signal processing and communication. The communication portal (128) facilitates data transmission between the device and hospital systems. The power source (130) provides energy for continuous operation. This embodiment details the electronic architecture of the device.

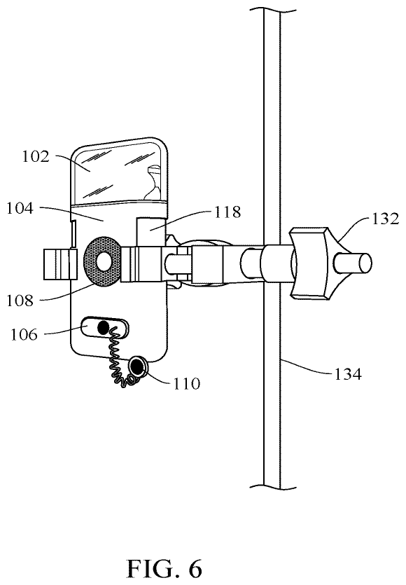

FIG. 6 illustrates how the device (100) is securely attached to an IV pole using a holding clip (132). The rod (134) provides additional support, ensuring stability when mounted on hospital equipment. This embodiment demonstrates the adaptability of the device for different hospital settings, allowing easy placement in various patient care environments.

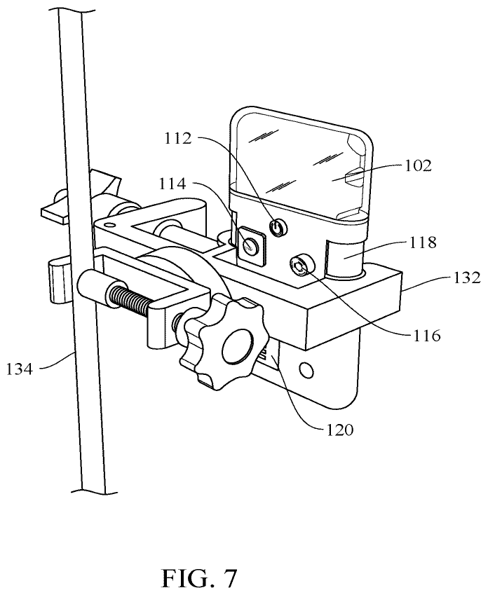

FIG. 7 illustrates a detailed view of the patient-accessible controls. The patient call button (106) is designed to be easily pressed by the patient when assistance is needed. The speaker (108) is integrated into the device to enable real-time voice communication. This embodiment highlights how the device ensures seamless patient-to-staff interaction.

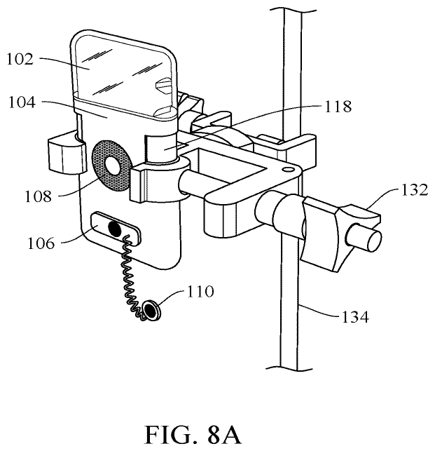

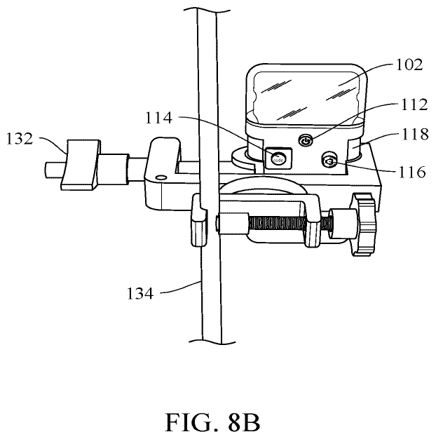

FIGS. 8A and 8B focuses on the emergency response features of the device (100). The code blue button (114) is positioned to allow medical staff to activate a critical alert when an emergency occurs. The rapid response button (116) serves as a secondary alert system for urgent but non-life-threatening situations. This embodiment enhances hospital emergency protocols by providing a quick and efficient alert mechanism. The power button (112) located on the back of the device. This button allows hospital personnel to manually tum the device on or off. This embodiment ensures that the device can be quickly powered on or reset as needed.

The battery charging base, which is designed to accommodate multiple devices simultaneously. The charging base ensures that spare batteries remain fully charged and ready for use, allowing seamless operation without interruptions. This embodiment supports continuous device availability in busy hospital environments.

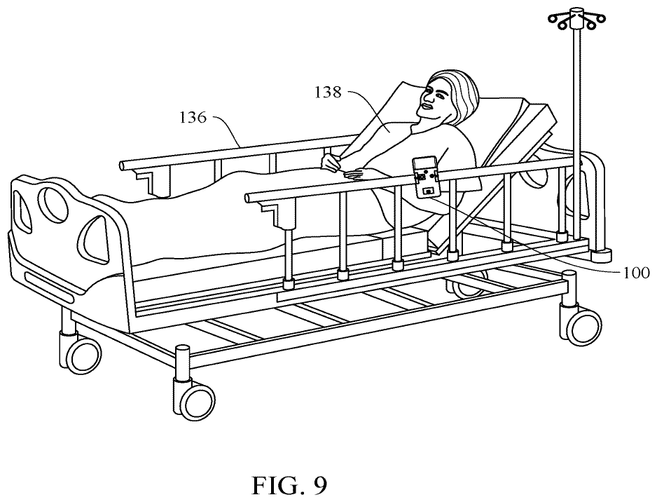

FIG. 9 illustrates the wireless call light device (100), in embodiments, may be mechanically coupled to, but not limited one or more patient bed (138). For example, as shown in FIG. 6, the device (100) may include the holding clip (132). The holding clip (132), in embodiments, may attach to, but not limited an IV pole or rod (136). The device 100 may be mechanically coupled to a wheelchair and/or a bed.

The adjustable clamp mechanism used to secure the device (100) to various hospital equipment. The clamp ensures stability while allowing easy repositioning. This embodiment enhances the versatility of the device by enabling attachment to different structures such as hospital beds, IV poles, and medical carts.

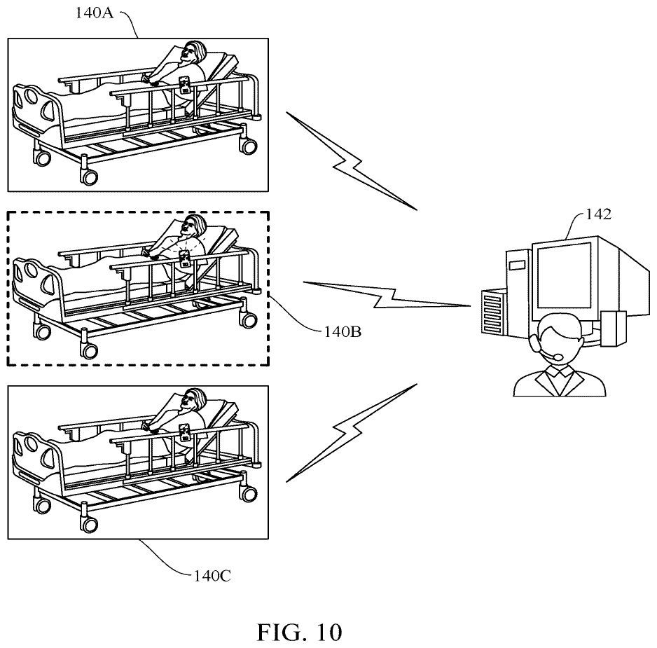

FIG. 10 is a block diagram illustrating a wireless call light device according to the present invention. This device is designed to enable wireless communication within a hospital and assigns an ID to each bed (140A-140C) for each floor and/or room arranged in a ward. When a call is made via the wireless button (106), the call signal is received through the central communication module (142), and the module displays the entire ward and highlights the bed (140B) that sent the call signal on the monitor. The module includes a main server, typically installed in a location such as the nurse's room, which is configured to transmit the call signal—formatted as a text message—to the nurse and/or doctor in charge, based on the ID of the call signal.

The registration process for the device (100) when assigned to a patient. When a nurse retrieves the device, the power it on using the power button (112). The device prompts for an assigned bed number, which is input via a voice system. Once the device is successfully registered, it announces its identification, such as “Hallway 5,” and is ready for use. This embodiment details the ease of setting up and configuring the device in a hospital environment.

The call signal's ID is unique for each device and each bed (140A-140C) within the room, allowing the central communication module (142) to identify the room and bed (140A-140C) when the call signal is received. This feature enables nurses and doctors to immediately identify the calling patient, thereby facilitating prompt treatment.

The central communication module (142) is located in the nurse's room, is programmed to display a pop-up window on the monitor according to the call signal. It shows the ward number, bed position (such as patient name), and other details based on the ID of the incoming call.

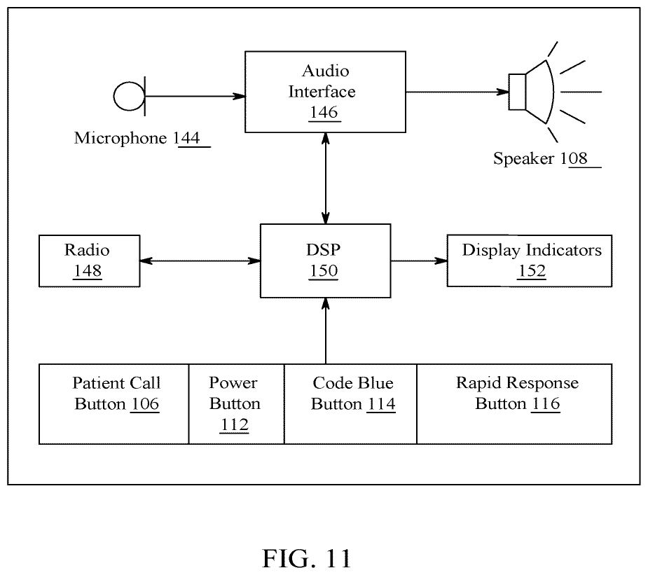

FIG. 11 illustrates a comprehensive diagram of the electronic systems integrated into the device (100). The microphone (144) captures patient audio input. The audio interface (146) processes voice signals for communication clarity. The speaker (108) outputs the staff's response. The radio (148) facilitates wireless data transmission, while the digital signal processor (DSP) (150) optimizes audio quality. This embodiment provides a technical overview of how different electronic components interact within the system.

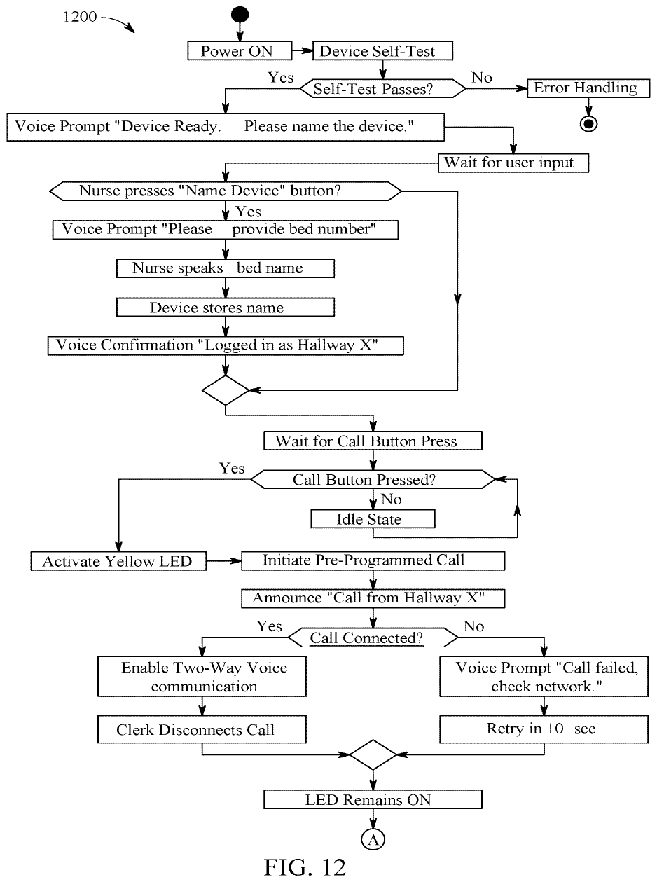

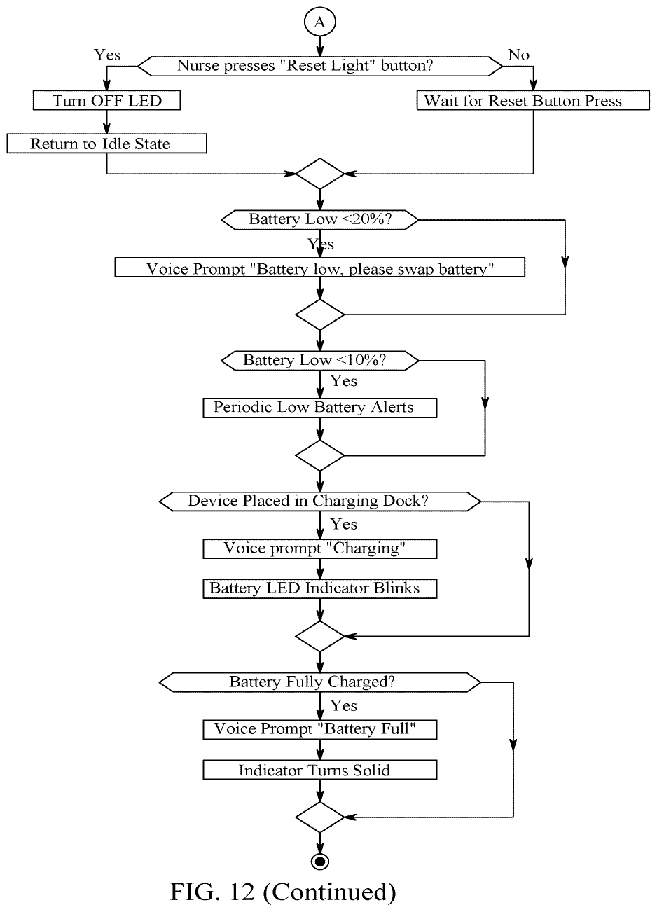

FIG. 12 illustrates a process flow for how the device (100) operates. When the patient call button (106) is pressed, a signal is sent via the communication portal (128) to the hospital's central system. The indicator light (102) is activated, and an audio connection is established via the speaker (108). The staff member at the central system responds through the device, enabling real-time communication. The light remains on until manually turned off by medical personnel. This embodiment illustrates the step-by-step process of how the system functions in a hospital setting.

When the device is initially set up, it must be powered on by the medical staff using the power button. The device will emit a confirmation sound or indicator light to indicate that it is ready for use.

The nurse or medical staff will then assign a name to the device, corresponding to the patient's bed number or location (e.g., Hallway 5, Room 103, etc.). This step ensures that when the call is received, the staff member knows exactly where the call is coming from.

The device is equipped with an adjustable clamp or mounting bracket, allowing the staff to attach the device securely to the hospital bed, IV pole, or gurney. The flexible cord attached to the patient's call button allows the patient to have easy access to the button wherever they are positioned.

The call button can either be directly mounted on the device or placed on the flexible cord for ease of access. The speaker and microphone are built into the device and remain in close proximity to the patient during use.

The patient will press the call button to request assistance. Once the button is pressed, the indicator light on the device will tum on, and a signal will be sent to the communication portal. The light will remain on until it is manually deactivated by a staff member, ensuring that the request for help is visible to those in the vicinity, even if they are not directly near the device.

Simultaneously, the communication portal will display the patient's location, and medical staff will receive the alert. If necessary, the system can also alert specific medical personnel based on the severity of the request (e.g., code blue for emergencies or rapid response for urgent but non-critical issues).

Once the call is received at the communication portal, the medical staff can respond to the patient via the speaker and microphone on the device. This allows for two-way communication, where the healthcare provider can inquire about the patient's needs, provide instructions, or assess the situation.

The volume and clarity of communication are adjustable through the audio interface. Noise-canceling features ensure that the conversation remains clear even in noisy hospital environments.

If the situation is critical, the medical staff can press the code blue button or the rapid response button. This will activate a flashing blue or red light on the device and send an emergency signal to the communication portal, alerting the appropriate team.

The code blue button is specifically designed for emergency situations that require immediate intervention, such as cardiac arrest or other life-threatening conditions. The rapid response button is used for situations that are urgent but do not require the immediate, full-scale response associated with a code blue.

The device includes a battery compartment that must be checked regularly to ensure that the device is powered adequately. The rechargeable battery is designed to last through an entire shift, but staff must be vigilant about checking the battery life to ensure that it remains operational.

When the battery is low, the system will send a notification to the communication portal, alerting the staff to recharge or replace the battery as necessary. By following these steps, the wireless communication system ensures a smooth and efficient process for requesting and providing assistance in a hospital setting. The system is designed to be adaptable, easy to use, and reliable, making it a crucial tool for improving patient care and hospital workflows.

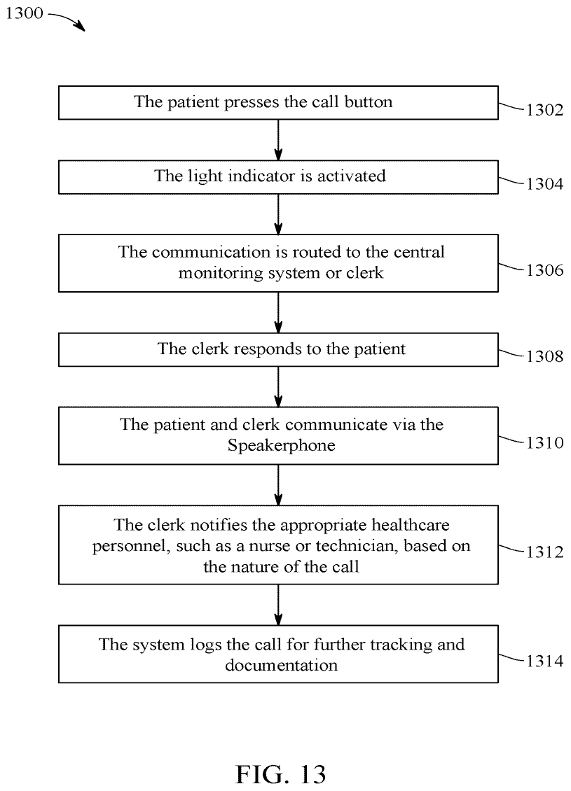

FIG. 13 illustrates two-way communication of the invention. Upon activation of the call button, an LED light is illuminated, signaling that the patient is requesting assistance. The light remains on until manually turned off by a medical staff member. This ensures that the call is not ignored or missed, even if hospital staff is not immediately near the device. The persistent visual indicator makes the communication system more reliable, especially in environments where patients may be out of direct line of sight of staff

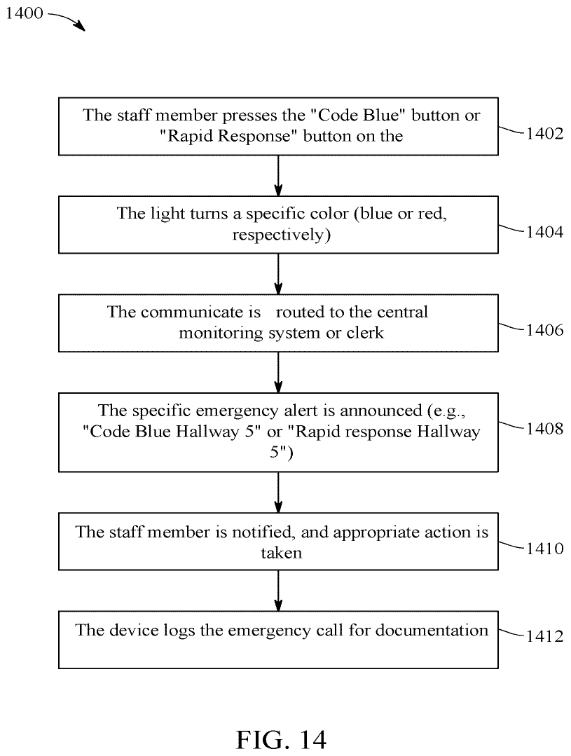

FIG. 14 illustrates a separate flowchart demonstrating how the code blue button (114) and rapid response button (116) function. When a staff member presses the code blue button (114), the device automatically places a high-priority alert to hospital emergency teams. Similarly, pressing the rapid response button (116) sends an urgent but lower-priority alert. This embodiment emphasizes how the device enhances emergency response workflows in hospitals.

From FIG. 12-14, the wireless communication system works by creating a two-way communication channel between the patient and the medical staff. Below is a step-by-step breakdown of how the system operates:

Device Activation Upon patient placement in a hallway bed (136), a nurse or medical staff member assigns the device to the patient's bed. The patient call button (106) is mounted either on the device or on a flexible cord (110) that can be easily placed in the patient's reach. The nurse powers on the device by pressing the power button (112), and the system prompts the nurse to assign the device a name corresponding to the patient's bed number. For example, the nurse could name the device “Hallway 5” for a patient in bed 5. Once the name is confirmed, the device is ready for use.

Patient Call When the patient requires assistance, they simply press the patient call button (106) or the flexible cord button (110). This action sends an alert to the hospital communication system (1302). The alert triggers several actions:

The indicator light (102) turns on, signaling the need for assistance (1304).

A call is placed to the clerk or central desk (via radio (148) or Wi-Fi communication) to notify them of the patient's request (1306).

The patient's name and assigned bed number are announced to the clerk, such as “Call from Hallway 5.”

Communication with Medical Staff Once the call is received, the clerk can communicate with the patient using the built-in speaker (108) and microphone (144) on the device. This allows for clear and immediate communication, without the patient needing to shout or rely on staff hearing their verbal requests. The clerk can then relay the message to the appropriate medical personnel, such as a nurse or technician (1308-1314).

Urgent Requests for emergency situations, the patient or staff can press the Code Blue (114) or Rapid Response (116) buttons (1402). Upon activation:

The light on the device will flash the respective color (blue for Code Blue, red for Rapid Response) (1404).

A call will be made to the clerk desk, and the emergency message will be announced, such as “Code Blue Hallway 5” or “Rapid Response Hallway 5.” (1406-1408)

The staff is immediately made aware of the urgent situation and can proceed with an appropriate response (1410).

After the call has been placed, the indicator light remains on until manually turned off by a staff member. This feature ensures that the request is not overlooked, even if the clerk or nurse is occupied with other tasks. The manual override process is simple, requiring the staff to press the light on/off button located on the device.

The device is powered by a swappable battery located in the battery compartment (120). The swappable battery ensures that the device can be quickly recharged and ready for use. A charging base allows the device to be docked and charged when not in use, ensuring no downtime for critical communication.

### Advantages of the Invention

The wireless communication system offers numerous advantages over traditional patient call systems, particularly in hospital emergency departments or temporary patient beds in hallways.

Increased Safety and Efficiency by enabling wireless communication between the patient and hospital staff, the system reduces response times, ensures that patient requests are not overlooked, and enables medical staff to respond more quickly to urgent needs.

The simplicity of pressing a button to call for assistance and the ease with which the device can be assigned to a patient ensures that it is user-friendly for both patients and staff

The portable nature of the device allows it to be used in temporary bed settings or hallways, which may not be served by traditional call light systems. Additionally, the device can be clipped to various locations within the hospital.

The inclusion of two-way audio communication ensures that patients and medical staff can communicate directly without unnecessary delays or misunderstandings. This is especially important in time-sensitive medical situations.

The ability to call for help quickly and directly reduces patient anxiety and improves their overall experience in the hospital. This invention provides a novel solution to an existing problem in the healthcare industry. By combining wireless communication, visual alerts, and audio interfaces, the device enhances the efficiency and safety of emergency and non-emergency communication in hospitals. The system's user-friendliness, flexibility, and reliability make it an essential tool for improving patient care and staff workflow in healthcare environments.

This invention provides a novel solution to an existing problem in the healthcare industry. By combining wireless communication, visual alerts, and audio interfaces, the device enhances the efficiency and safety of emergency and non-emergency communication in hospitals. The system's user-friendliness, flexibility, and reliability make it an essential tool for improving patient care and staff workflow in healthcare environments.

The wireless communication system addresses a significant need for more effective communication between patients and healthcare providers, particularly in hospitals where rapid responses are often required. In such settings, every second counts when patients need medical attention, and current communication methods, which typically involve wired intercom systems or phone calls, can be inefficient and prone to delays. This invention eliminates those inefficiencies by allowing patients to make a direct and immediate request for assistance through an easy-to-use call button. The system is designed to be intuitive, making it simple for patients to signal for help, regardless of their physical or cognitive limitations.

One of the key features of the invention is the use of an indicator light that provides a visual signal whenever a patient has requested assistance. The light turns on as soon as the call is placed and remains on until manually turned off by the medical staff This feature helps ensure that hospital staff are immediately aware of the patient's request, even if they are engaged with other tasks or located in different areas of the hospital. This continuous visual cue eliminates the need for staff to constantly monitor a screen or a phone for incoming requests, reducing response time and improving the overall workflow.

The system is equipped with multiple patient call buttons, which can be mounted either directly on the device or attached to a flexible cord for easy accessibility. This flexibility ensures that the patient can choose the most comfortable and convenient location for the call button, whether it's attached to the hospital bed or held in hand. When the patient presses the button, a signal is sent wirelessly to the hospital's central communication system, notifying medical staff of the need for assistance. The wireless nature of the system makes it adaptable to different hospital layouts, ensuring that communication can take place from any location within the hospital without the need for physical wiring.

The device's two-way communication feature allows patients to not only request help but also communicate directly with medical staff The system includes a speaker and microphone, enabling the staff to listen to the patient's needs and respond promptly. This audio interface ensures that the patient's concerns are heard clearly, even in noisy environments like hospital hallways or emergency rooms. The system is equipped with noise-canceling technology that filters out background noise, making communication clear and efficient. Additionally, the volume of the speaker can be adjusted automatically based on the surrounding noise levels, ensuring that the patient's voice can always be heard.

To further improve the system's functionality, the invention includes a power button that enables medical staff to turn the device on and off This button is designed to be easy to locate and press, even when the staff is wearing gloves or engaged in other tasks. The power button is an essential feature for ensuring the device operates only when needed, helping to conserve battery life and prevent accidental activations.

The device's battery compartment houses a rechargeable battery that provides continuous power for the system. The battery is designed to last through an entire shift, ensuring that the device remains operational without the need for frequent recharging. When the battery runs low, the system includes an alert mechanism to notify staff that it's time for recharging. Additionally, the device is designed to be easily placed in a charging station when not in use, allowing it to be quickly recharged and ready for the next use. This self-contained power management system eliminates the need for external power sources or complicated setups, making the device fully portable and versatile.

The communication portal serves as the central hub for managing patient requests and directing them to the appropriate medical staff When a call is made by a patient, the communication portal receives the signal and forwards it to the designated staff member, such as a nurse or doctor, who is best equipped to address the patient's needs. The portal operates on a wireless communication network, such as Wi-Fi or radio frequency, ensuring reliable transmission of the patient's request. This system can be integrated into existing hospital infrastructure, seamlessly working with other communication systems, such as nurse call systems or emergency response systems.

To ensure the system is easy to use, the interface is designed with simplicity in mind. The indicator light provides a clear visual cue for staff, while the speaker and microphone allow for clear two-way communication between patients and staff The device can be mounted in various ways, allowing it to be placed near the patient's bed, on a gurney, or on an IV pole. The mounting system includes adjustable clips and brackets, making it easy to position the device in a way that's most convenient for the patient and staff.

The system is also designed for easy maintenance. The battery compartment can be accessed quickly for battery replacement or servicing, and the components are modular, making it easy to repair or replace individual parts. The entire system is built with durability in mind, using high-quality materials that can withstand the rigors of a hospital environment. The components are designed to be easy to clean, ensuring that infection control protocols are met.

In terms of scalability, the system can be expanded to accommodate larger healthcare facilities. Multiple communication portals can be set up across the hospital, ensuring that all patient requests are efficiently routed to the appropriate staff The system is flexible enough to be adapted to various hospital layouts, whether for a small clinic or a large multi-building hospital complex. The wireless nature of the system allows it to be easily expanded without the need for extensive rewiring or infrastructure changes.

Furthermore, the system can be integrated with other hospital systems to improve its functionality. For instance, it can be connected to the hospital's electronic health records (EHR) system, allowing staff to access patient information and medical histories in realtime. This integration can help staff provide more accurate and timely care, as they'll have all the necessary information at their fingertips when responding to a patient's request.

The device also features a manual override function for the indicator light. Once the patient's request is addressed, the medical staff can manually turn off the indicator light, signaling that the issue has been resolved. This simple yet effective feature ensures that staff can maintain control over the system while minimizing the risk of confusion or redundant calls.

The wireless communication system revolutionizes patient care in healthcare environments. By combining visual, audio, and wireless technologies, the device streamlines communication between patients and healthcare providers, improving both the efficiency of staff workflows and the quality of patient care. The system's portability, ease of use, and integration with existing hospital systems make it a versatile and essential tool in modern healthcare. This invention not only enhances patient safety by ensuring timely responses but also helps medical staff manage their tasks more effectively, ultimately leading to better patient outcomes and a more efficient hospital environment.

The device consists of several components that must be assembled carefully to ensure functionality and durability. The key parts of the device are, but not limited to the indicator light, enclosure (case), patient call button, speaker, power button, code blue button, rapid response button, battery compartment, communication module, and communication portal. These components work together to form a complete and integrated system that enables communication between patients and healthcare providers.

The enclosure of the device is the main body, designed to protect the internal components. Itis typically made from durable plastic or other high-quality, medical-grade materials that are easy to clean and resistant to the harsh conditions of a hospital environment, including disinfection agents. The enclosure is designed with a compact form factor to be easily mounted or placed in various positions near the patient's bed or on an IV pole. The device's design incorporates a mounting bracket or clip that allows for secure attachment to a variety of surfaces, ensuring flexibility in deployment.

The indicator light is placed on the front or top of the device for visibility. The light turns on when the patient presses the call button, serving as a visual alert for staff that assistance is required. The light stays on until the medical staff manually deactivates it. This feature ensures that even if a staff member is not immediately available to answer the call, others in the area can see that assistance is needed. The light must be bright enough to be visible even from a distance or in low-light conditions.

The patient call button is located on the front of the device. It is designed to be large and easy to press, ensuring that patients, even those with limited mobility, can easily request assistance. The button is a simple, pressure-sensitive switch that sends a wireless signal to the communication portal when pressed. This button can be mounted on the main device or attached to a flexible cord, allowing the patient to place it wherever is most convenient, such as on the bed frame or IV pole. When the patient presses the button, the call is triggered, sending a signal to the communication portal and activating the indicator light.

The speaker is used for two-way communication between the patient and the staff. It is integrated into the device, with the microphone allowing the patient to speak to the healthcare provider, and the speaker allowing the healthcare provider to respond. The speaker is designed with noise-canceling technology to ensure clear communication in the noisy environment of a hospital. The volume can be adjusted automatically based on ambient noise levels to ensure the patient's voice is audible.

The power button is located on the device's exterior and is used to turn the device on or off. It is designed to be easy to use, even for staff wearing gloves or operating under time constraints. The button is placed in a convenient location, ensuring that staff can activate or deactivate the device without unnecessary effort. When the device is powered on, it is ready to receive calls from patients and communicate with the central communication portal.

The code blue button is a special feature designed for emergencies. This button sends a call to the central desk with the message “Code Blue” followed by the patient's bed number or location. The light on the device will flash blue, and a specific emergency protocol is activated at the healthcare facility. This button is located on the back of the device to prevent accidental activation by patients. The placement of the button ensures that only medical staff can trigger a code blue call.

The rapid response button functions similarly to the code blue button, but it is used in situations that require urgent attention but are not critical emergencies. When pressed, the button sends a signal to the communication portal with the message “Rapid Response” followed by the patient's bed number or location. The light on the device will flash red, signaling that urgent but non-emergency assistance is needed. Like the code blue button, the rapid response button is located on the back of the device, preventing accidental presses by patients.

The battery compartment houses the rechargeable battery that powers the device. The battery should be capable of lasting through a full shift (typically eight hours) without needing to be recharged. The battery is designed to be easily replaceable, ensuring the device remains operational throughout the day. The battery is accessed via a compartment that can be opened for replacement or recharging. The rechargeable battery is capable of retaining power for an extended period, reducing the need for frequent charging.

The communication module is the heart of the device, providing wireless connectivity to the central communication portal. The communication module uses Wi-Fi or other wireless communication protocols (such as Bluetooth or radio frequency) to send signals between the patient's device and the healthcare staff s central communication system. This allows for immediate transmission of the call to the appropriate nurse, doctor, or emergency response team. The wireless nature of the device eliminates the need for complicated wiring, making it adaptable to various hospital layouts.

The communication portal serves as the interface for healthcare providers to receive notifications from the wireless call buttons. When the patient presses a button on the device, the communication portal receives the signal and alerts the designated medical staff The portal is typically located at the nurse's station or other central location within the hospital. The portal can be integrated with other hospital systems, such as the electronic health record (EHR) system or emergency response protocols, allowing staff to access important patient data and provide more accurate and timely care.

In the detailed description, the venous components of the wireless communication system, as shown in the accompanying drawings, are labeled using reference numerals for ease of identification. These reference numerals allow for a direct correlation between the functional description and the physical elements illustrated in the drawings.
Indicator Light (102)—The indicator light serves as a visual alert to signal the status of a call request. It turns on when the patient presses the call button and stays on until manually deactivated by the medical staff. This feature ensures that the healthcare staff is clearly notified when assistance is needed, even if the staff member is not immediately available to respond.
Enclosure/Case (104)—The enclosure, or main body of the device, houses the internal components of the wireless communication system. The enclosure protects the delicate electronics and is designed to withstand the harsh conditions of a hospital environment, such as frequent disinfection, drops, or accidental exposure to liquids.
Patient Call Button (106)—The patient call button is a key element of the system that allows patients to request assistance. Located on the device, it is activated when pressed and sends a signal to the communication portal, triggering the indicator light to tum on. An alternative design allows the patient call button to be mounted on a flexible cord (110), making it more convenient for patients to access from various locations.
Speaker (108)—The speaker is integrated into the device and is used for two-way communication between the patient and the medical staff It allows the healthcare provider to communicate with the patient, responding to their request for help or offering instructions.
Power Button (112)—The power button is used to turn the device on or off This button must be easily accessible to medical staff and is designed to be operable even when staff are wearing gloves or are in a hurry.
Code Blue Button (114)—The code blue button is a specialized feature that allows staff to initiate an emergency alert. When pressed, the button sends an urgent signal to the communication portal and triggers the flashing blue light to indicate that immediate medical assistance is required.
Rapid Response Button (116)—Similar to the code blue button, the rapid response button is used for urgent situations but not for life-threatening emergencies. It triggers a rapid response protocol and flashes a red light, allowing medical staff to respond quickly without the need for a full-scale emergency response.
Grip (118)—The grip is an ergonomic feature designed to make it easier for healthcare providers to hold or mount the device. It provides a secure hold for medical staff when carrying or repositioning the device.
Battery Compartment (120)—The battery compartment houses the rechargeable power source that ensures the device remains operational. The compartment is designed to be easily accessible for maintenance or replacement of the battery as needed.
Portable Electronic Device (122)—The portable electronic device encompasses all the features necessary to operate the system, including the call button, speaker, power supply, and communication module. The device is lightweight and portable, making it easy to transport within the hospital environment.
Memory (124)—The memory element stores configuration data and call logs for the system. It ensures that the device can be configured to meet the specific needs of each patient and healthcare setting.
Processor (126)—The processor manages the internal operations of the device, including signal processing, communication with the communication portal, and controlling the indicator light. The processor enables the device to function efficiently and reliably.
Communication Portal (128)—The communication portal serves as the central interface where medical staff receive alerts from the wireless call buttons. It is connected to the device via wireless communication protocols, allowing real-time notification and two-way communication.
Power Source (130)—The power source provides the necessary energy to operate the wireless communication system. It may consist of a rechargeable battery, designed to provide long-lasting power, or an alternative power solution, depending on the application.
Holding Clip (132)—The holding clip is used to attach the device securely to the hospital bed, IV pole, or other suitable locations. It ensures the device remains in place during use, preventing it from being accidentally dislodged.
Rod (134)—The rod is a flexible element that can be adjusted to allow for easy positioning of the patient call button. The rod is attached to the device and can be clipped to the patient's bed, ensuring that the call button is within easy reach of the patient.
Hospital Bed (136)—The hospital bed provides the platform for attaching the device, ensuring that it is within the patient's reach. The device is mounted securely to the bed, enabling easy access to the call button.
Patient (138)—The patient interacts with the device by pressing the call button when they need assistance. The patient's comfort and ease of use are considered during the design of the system, ensuring that they can operate the device even if they have limited mobility.
Hospital/Patient System (140)—The hospital/patient system integrates the wireless communication device with the healthcare facility's central systems. It ensures that patient requests are properly routed to the appropriate staff members and that the responses are logged for future reference.
Central Database System (142)—The central database system stores and manages the data collected from patient requests, ensuring that records are kept for analysis and performance monitoring. The database may be accessed by medical staff for historical reference or to identify patterns in patient needs.
Microphone (144)—The microphone is part of the two-way communication system. It picks up the patient's voice and transmits it to the medical staff via the speaker, enabling clear and effective communication.
Audio Interface (146)—The audio interface manages the sound levels for both the speaker and microphone, ensuring that the voice transmission is clear and audible, even in noisy environments.
Radio (148)—The radio module enables wireless communication between the device and the central communication portal. Ictuses standard wireless communication protocols to ensure reliable signal transmission.
DSP (150)—The digital signal processor (DSP) manages the data signals, ensuring clear communication between the device and the healthcare staff The DSP also handles noise filtering, improving the clarity of the audio signal.
Display Indicators (152)—The display indicators provide visual feedback to the healthcare staff regarding the status of the call. They may show when a call is active or indicate the priority level of the call, such as emergency or rapid response.

There are several alternative embodiments or variations of the invention that can be used to customize the system for different healthcare settings or requirements. Some of these variations include different communication protocols, alternative materials for the device housing, and optional features such as additional sensors or environmental controls.

Communication Protocols: While the invention primarily uses wireless communication methods such as Wi-Fi or Bluetooth, alternative communication technologies such as ZigBee, Lora, or cellular networks can be used depending on the hospital's existing infrastructure and requirements. These alternative protocols may offer longer ranges or more energy-efficient communication, making them suitable for larger healthcare facilities or rural hospitals.

Alternative Housing Materials: The enclosure (104) of the device is typically made from high-quality plastic materials that are durable and easy to clean. However, other materials, such as antimicrobial coatings or medical-grade metals, may be used to enhance the device's durability and resistance to germs and contamination. These alternatives can be selected based on the specific needs of the healthcare environment.

Additional Sensors and Environmental Controls: In some variations, the wireless communication system may be equipped with additional sensors, such as temperature sensors, motion detectors, or environmental monitoring systems. These sensors can provide real-time data to the medical staff, allowing them to monitor the patient's environment and respond to changes in their condition. Additionally, the system may be equipped with environmental controls to adjust lighting or temperature in the patient's room based on their needs.

Multiple Communication Channels: The system maybe expanded to include multiple communication channels, allowing for more complex interactions between patients and medical staff for example, the system may incorporate video communication capabilities, enabling doctors to visually assess the patient's condition remotely.

The materials and methods used in the invention are critical to ensuring the device's reliability and safety in a hospital environment. The device must be durable, easy to clean, and resistant to contamination.

Materials Used for the Housing and Components: The housing (104) of the device is typically made of medical-grade plastic, such as ABS or polycarbonate, which offers strength, resistance to impact, and ease of cleaning. The plastic is treated with antimicrobial agents to reduce the risk of bacterial growth. Additionally, the material is non-porous, making it easy to wipe down with hospital-grade disinfectants. The components, such as the patient call button (106), speaker (108), microphone (144), and indicator light (102), are made from high-quality, durable materials designed to withstand the demanding environment of a hospital. The electronic components are housed in the enclosure to prevent exposure to dust, moisture, and other contaminants.

Assembly and Manufacturing Methods: The device is assembled in a controlled environment, ensuring that each component is tested and verified before being placed into the final assembly. The communication module (128) is calibrated to ensure reliable wireless communication between the device and the central communication portal (140). The power source (130) is integrated with the system to ensure the device remains operational throughout a typical hospital shift.

Quality Control: During the assembly process, each unit undergoes rigorous quality control tests to ensure functionality, durability, and compliance with medical device standards. This includes testing for signal strength, communication reliability, battery life, and ease of use.

The invention provides a reliable, easy-to-use system for improving communication and patient care in healthcare settings. Each element is designed to work together seamlessly to ensure the safety, comfort, and well-being of patients while enhancing the efficiency of healthcare providers.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclosed herein suitably may be practiced in the absence of any element which is not specifically disclosed herein—and in particular, embodiment specifically contemplated, is intended to be practiced in the absence of any element which is not specifically disclosed herein.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

While this invention has been described in conjunction with the exemplary embodiments outlined above, it is evident that many alternatives, modifications and variations will be apparent to those skilled in the art. Accordingly, the exemplary embodiments of the invention, as set forth above, are intended to be illustrative, not limiting. Various changes maybe made without departing from the spirit and scope of the invention. Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

### AI WIRELESS CALL LIGHT DEVICE

In some embodiments, a wireless call light device is provided for use in a healthcare or assisted-care environment to enable a patient or other user to initiate assistance requests, communications, or alerts to a receiving system or caregiver. The device may be configured for mounting to or association with a patient support apparatus, including a hospital bed, stretcher, wheelchair, or IV pole. The wireless call light device may comprise an enclosure housing electronic components including a processor, memory, communication circuitry, audio components, user input interfaces, and a power source. The device may further include one or more output devices configured to provide visual, audio, and tactile feedback to a patient.

In some embodiments, the wireless call light device comprises a communication portal configured to place a call or transmit an assistance request to a pre-programmed recipient upon activation of a call initiation control. The communication portal may include wired or wireless communication circuitry, including Wi-Fi, Bluetooth, Bluetooth Low Energy (BLE), cellular communication, proprietary radio-frequency communication, Ethernet, or hospital network communication protocols. In certain embodiments, the communication portal transmits signals to a central database system, nurse station, mobile device, server, or cloud-based receiving system. The communication portal may be configured to transmit calls, alerts, coded event signals, or data packets corresponding to patient requests.

In some embodiments, the call initiation control comprises a physical input interface mounted on the enclosure of the device, including a button, capacitive touch surface, or pressure-sensitive input. In other embodiments, the call initiation control comprises a remotely coupled input device including a flexible cord and a grip portion configured for patient accessibility. The flexible cord may be detachably or permanently coupled to the enclosure and may permit activation by patients with limited mobility. In further embodiments, patient input may be received via a non-physical interface, including voice input processed by an artificial intelligence interface, such that either physical or AI-interpreted input is sufficient to initiate an assistance request.

In some embodiments, the wireless call light device includes a speaker and a microphone configured to enable two-way audio communication between a first user and a second user. The speaker and microphone may be integrated into the enclosure or externally coupled to the device. In certain embodiments, the audio components are activated automatically upon initiation of a call or upon acceptance of the call by a receiving system.

In some embodiments, the wireless call light device includes a memory and a processor configured to store and execute instructions. The memory may store a user-programmable identifier corresponding to a patient support apparatus, room number, bed number, or device identifier. In certain embodiments, when a call is placed, the processor transmits the stored identifier to a central database system or receiving system, enabling accurate location identification of the requesting patient.

In some embodiments, the device includes an indicator light configured to illuminate when a call or assistance request is initiated. The indicator light may remain illuminated until manually turned off via an authorized reset mechanism. In certain embodiments, the processor places the device into a latched call state upon call initiation, wherein repeated activation of the call initiation control is suppressed until receipt of an authorized reset command. The reset command may be generated locally via a staff-only input, remotely via the receiving system, or automatically upon acknowledgment of the request.

In some embodiments, the wireless call light device includes a power source comprising a battery housed within a battery compartment. In certain embodiments, the battery is hot-swappable such that battery replacement may occur without interrupting device operation. In some embodiments, the device includes a holding clip, clamp, or rod attachment mechanism configured to secure the device to a patient support apparatus, rail, frame, or IV pole.

In some embodiments, the wireless call light device includes an artificial intelligence interface executed by the processor and operatively coupled to patient-facing software functions and controllable hardware components of the device. The artificial intelligence interface may be configured to receive patient input, including voice input, interpret patient intent or assistance requests, and determine one or more device actions to be performed.

In certain embodiments, the artificial intelligence interface comprises an artificial intelligence processing module configured to operate locally on the device, remotely via a cloud-based system, via a centralized local processing hub, or any combination thereof. The artificial intelligence interface may process captured voice signals to recognize impaired, unclear, non-standard speech patterns, or non-word vocalizations, and may convert patient input into standardized assistance events for transmission to a receiving system.

In some embodiments, standardized assistance events represent one or more event types including assistance request events, communication request events, general alert events, emergency alerts, rapid response alerts, or code blue alerts. In certain embodiments, the artificial intelligence interface determines a confidence level associated with interpretation of patient input. The confidence level may be generated using probabilistic models, pattern recognition algorithms, or machine learning classifiers.

In some embodiments, when the confidence level fails to satisfy a predetermined threshold, including a selectable threshold of eighty percent or less, the device enters a fallback mode. In fallback mode, the device generates an assistance request, transmits an indication that interpretation confidence is limited, and transmits recorded audio input or text-based representations of the patient input to a receiving system. In some embodiments, the wireless call light device supports both physical input controls and voice-based input for initiating predefined alert types. The artificial intelligence interface is configured to operate as an alternative input mechanism to existing physical buttons, such that verbal commands detected by the artificial intelligence interface correspond to the same alert categories previously associated with the physical input controls.

In some embodiments, verbal commands detected by the artificial intelligence interface are mapped to predefined alert functions already supported by the device, including emergency alerts, rapid response alerts, or code-related alerts, in the same manner as activation of corresponding physical buttons. The artificial intelligence interface does not create new alert types, but instead selects from alert types already defined for the device. In some embodiments, the artificial intelligence interface is configured to analyze captured audio to detect predefined trigger phrases, keywords, or vocal patterns corresponding to existing alert functions. Detection may be based on speech recognition, keyword spotting, pattern matching, probabilistic classification models, or combinations thereof executed by the processor.

In certain embodiments, a detected verbal command corresponding to an emergency alert may include phrases such as “help,” “I need help now,” or “emergency assistance.” In certain embodiments, a detected verbal command corresponding to a rapid response alert may include phrases such as “rapid response,” “medical team needed,” or “urgent assistance.” In certain embodiments, a detected verbal command corresponding to a code-related alert may include phrases such as “code blue,” “cardiac emergency,” or “call a code.” n some embodiments, upon detecting a verbal command corresponding to an emergency alert, rapid response alert, or code-related alert, the wireless call light device is configured to require a confirmation step prior to initiating the alert. The confirmation step reduces false activations and ensures that alert initiation reflects intentional user input.

In some embodiments, the confirmation step includes generating an audible prompt requesting confirmation, detecting a repeated verbal command within a predetermined time window such as 30 seconds, receiving an affirmative verbal response, receiving a secondary physical input. For example, the device may prompt, “Do you want to initiate a Code Blue?” and proceed only after detecting a verbal confirmation such as “yes” or “confirm.” n some embodiments, if confirmation is not received or if interpretation confidence fails to satisfy a predetermined threshold, the device refrains from automatically initiating the alert and instead enters a fallback mode. In fallback mode, the device transmits captured audio and an indication of limited confidence to a receiving system for human review and determination of the appropriate response. In all embodiments, the artificial intelligence interface does not determine, diagnose, or confirm the existence of a medical condition and does not provide clinical decision support. The artificial intelligence interface merely interprets user input to identify a requested alert type, and initiation of any alert occurs only after confirmation, authorization, or human review consistent with predefined device control logic.

In some embodiments, the foregoing voice-based alert initiation embodiments are functionally equivalent to embodiments in which the alert is initiated by activation of a corresponding physical input control. These embodiments are provided as alternative mechanisms for initiating predefined alert functions without altering the underlying alert handling workflow, escalation logic, or responsibility of human personnel for medical interpretation and response.

In certain embodiments, recorded audio associated with patient input is automatically discarded when the confidence threshold is satisfied and retained or transmitted only when the threshold is not satisfied. In further embodiments, recorded audio is retained until acknowledgment of receipt is received from the receiving system, after which the audio is automatically discarded.

In some embodiments, the wireless call light device includes multimodal output to a patient, including visual output, audio output, and haptic output. The multimodal output may be generated in response to patient input, device state, system state, or operation of the artificial intelligence interface. In certain embodiments, the device further includes a display screen configured to present information to the patient, including call status, acknowledgments, or messages, and to provide output in coordination with audio and haptic feedback.

In some embodiments, interpretation of patient input is performed locally on the wireless call light device, and transmission to a remote system occurs only after generation of a standardized assistance event. In other embodiments, raw or partially processed input is transmitted to a remote system for interpretation.

In some embodiments, the wireless call light device is configured to communicatively couple with one or more other wireless call light devices or receiving systems. In certain embodiments, the device is configured to relay standardized assistance events generated by another wireless call light device when a direct communication path to a receiving system is unavailable.

In some embodiments, transmission of coded event signals includes acknowledgment monitoring and automatic retry logic. If an acknowledgment is not received within a predetermined time interval, the device automatically retransmits the assistance request or escalates the alert.

The embodiments described herein disclose concrete device structures, control logic, software-hardware interactions, and operational workflows sufficient to enable a person of ordinary skill in the art to make and use the wireless call light device without undue experimentation. Variations in communication protocols, artificial intelligence models, processing locations, and hardware configurations may be implemented without departing from the scope of the claims.

### Artificial Intelligence Interface Definition

In some embodiments, the device includes an artificial intelligence interface configured to process patient input and determine one or more response actions. As used herein, the artificial intelligence interface may comprise one or more computational models or systems, including but not limited to rule-based decision systems, machine-learning models, neural networks, deep learning models, transformer-based models, large language models, probabilistic models, statistical classifiers, or combinations thereof. The artificial intelligence interface may be configured to process spoken language, non-verbal vocalizations, button noting inputs, or combinations of inputs to determine device behavior.

### Coupling to Software and Controllable Hardware

In some embodiments, the artificial intelligence interface is operatively coupled to patient-facing software functions and controllable hardware components of the device, including one or more of the communication portal, speaker, microphone, indicator light, patient call button, and other output or input components. The artificial intelligence interface is configured to control operation of such software functions and hardware components by determining response actions based on patient input.

### Local, Cloud-Based, or Hybrid Implementation

In some embodiments, the artificial intelligence interface is executed locally on the device by a processor of the device. In other embodiments, the artificial intelligence interface is executed remotely on a networked computing system, server, or cloud-based platform in communication with the device. In still other embodiments, the artificial intelligence interface is implemented in a distributed manner across both local and remote computing resources, wherein a local model performs initial processing or default response actions and a remote model performs additional interpretation, refinement, or follow-up processing.

### Fail-Safe and Connectivity-Independent Operation (Strong for Hospitals)

In distributed embodiments, the device is configured to continue operating using local processing when remote connectivity is unavailable, thereby ensuring reliable operation of the call light device independent of network availability.

### Haptic Output Devices

In some embodiments, the wireless call light device includes one or more haptic output devices configured to provide tactile feedback perceptible to a patient. As used herein, a haptic output device may comprise any device capable of generating tactile feedback, including but not limited to vibration motors, eccentric rotating mass motors, linear resonant actuators, piezoelectric actuators, electroactive polymer actuators, ultrasonic haptic devices, or combinations thereof.

### Integration Within the Call Light Device

In some embodiments, the haptic output device is disposed within the enclosure of the wireless call light device and is operatively coupled to the processor. In such embodiments, the haptic output device may be mounted to an internal surface of the enclosure or integrated into a structural component of the device such that activation of the haptic output device generates vibrations or tactile feedback detectable by a patient through contact with the enclosure or an attached component.

### Remote or Patient-Worn Haptic Devices

In other embodiments, the haptic output device is provided in a remote device separate from the enclosure of the wireless call light device. The remote device may be configured to be worn by or placed on the patient, including but not limited to a wearable device, pendant, wristband, clip-on module, or handheld unit. The remote device may include one or more haptic output devices configured to generate tactile feedback in response to signals received from the wireless call light device.

### Wired and Wireless Coupling

In some embodiments, the haptic output device is operatively coupled to the wireless call light device via a wired connection. In other embodiments, the haptic output device is operatively coupled via a wireless communication link, including but not limited to Bluetooth, Bluetooth Low Energy, Wi-Fi, near-field communication, or other wireless communication protocols. In still other embodiments, a combination of wired and wireless connections may be used.

In some embodiments, wireless communication between the wireless call light device and a receiving system utilizes one or more short-range wireless communication protocols, including but not limited to Bluetooth, Bluetooth Low Energy (BLE), Wi-Fi, Zigbee, or other radio-frequency-based protocols. In some embodiments, data transmitted via the communication portal is encrypted using one or more encryption techniques to protect patient information and device communications. In some embodiments, the wireless communication includes acknowledgment signaling, retry logic, or both, such that transmitted messages are confirmed as received by the receiving system and retransmitted when acknowledgment is not detected within a predetermined time interval.

### Control by Processor or Artificial Intelligence Interface

In some embodiments, activation of the haptic output device is controlled by the processor of the wireless call light device or by the artificial intelligence interface executed by the processor. The haptic output device may be activated in response to one or more events, including initiation of a call, acknowledgment of the call by hospital staff, receipt of a response, or determination of a device action by the artificial intelligence interface.

### Visual Display Screen Embodiments

In some embodiments, the wireless call light device includes one or more visual display screens configured to present information to a patient or hospital staff. As used herein, a “visual display screen” may comprise any electronic display capable of presenting visual information, including but not limited to liquid crystal displays (LCD), light-emitting diode (LED) displays, organic light-emitting diode (OLED) displays, electrophoretic or e-ink displays, micro-LED displays, touch-sensitive displays, monochrome displays, color displays, segmented displays, or combinations thereof.

### Display Integration With the Device Housing

In some embodiments, the visual display screen is integrated directly into the enclosure of the wireless call light device. The display screen may be positioned on a front surface, side surface, top surface, or rear surface of the enclosure and may be mounted flush with, recessed within, or protruding from the enclosure. In such embodiments, the display screen is operatively coupled to the processor and configured to display information including, but not limited to, device status, call state, patient location, emergency indicators, battery level, staff acknowledgments, or system messages.

### Remote or External Display Devices

In other embodiments, the visual display screen is provided as a remote display device separate from the enclosure of the wireless call light device. The remote display device may be positioned near the patient, worn by the patient, or located at a caregiver workstation. The remote display device may comprise a tablet, handheld device, wearable display, bedside monitor, wall-mounted display, or other electronic display capable of presenting information generated by the wireless call light device.

### Wired and Wireless Coupling

In some embodiments, the visual display screen is operatively coupled to the wireless call light device via a wired connection. In other embodiments, the visual display screen is operatively coupled via a wireless communication link, including but not limited to Wi-Fi, Bluetooth, Bluetooth Low Energy, radio-frequency communication, near-field communication, or other wireless protocols. In still other embodiments, a combination of wired and wireless connections may be used.

### Control by Processor or Artificial Intelligence Interface

In some embodiments, content displayed on the visual display screen is controlled by the processor of the wireless call light device or by the artificial intelligence interface executed by the processor. The artificial intelligence interface may determine display content based on patient input, call priority, emergency status, staff acknowledgment, system state, or historical interaction data. The display may present dynamic content, alerts, prompts, confirmations, or instructions in response to determinations made by the artificial intelligence interface.

### Accessibility and Hearing-Impaired Communication

In some embodiments, the visual display screen is configured to display textual content, graphical content, images, animations, or video content to facilitate communication with patients who are hearing impaired or have limited auditory perception. The displayed content may include written messages, captions, prompts, instructions, alerts, confirmations, or prerecorded or dynamically generated video content.

In some embodiments, the visual display screen presents real-time or near real-time text translating audio communications from the receiving system, or text generated from voice input received by the microphone. In other embodiments, the visual display screen presents prerecorded or generated video content configured to convey instructions, status updates, or responses from hospital staff without reliance on audio output.

In some embodiments, the artificial intelligence interface or processor determines when to present textual or video-based communication based on patient preferences, detected patient needs, accessibility settings, or system configuration, thereby enhancing communication for hearing-impaired patients or patients who prefer visual communication. In some embodiments, the visual display screen is further configured to present display elements representing patient location information, emergency or alert indicators, staff acknowledgments, system status messages, and textual representations of audio detected in an environment of the wireless call light device. Patient location information may include a room identifier, bed identifier, hallway location, or other patient support apparatus identifier stored in memory and associated with the device. Emergency or alert indicators may include visual icons, color-coded indicators, or textual labels corresponding to assistance requests, communication requests, or alert states. Staff acknowledgments may include visual confirmation that a request has been received, accepted, or resolved by a receiving system or caregiver device.

In some embodiments, the artificial intelligence interface is configured to process audio signals captured by the microphone and to generate real-time or near real-time textual subtitles for display on the visual display screen. The subtitles may represent spoken words, partial phrases, or non-verbal vocalizations detected in the vicinity of the device. In such embodiments, the artificial intelligence interface performs speech recognition processing on captured audio to convert detected speech into text suitable for display to the patient or staff. In some embodiments, the artificial intelligence interface is further configured to distinguish between multiple recognized voices detected by the microphone. Voice differentiation may be performed using one or more speaker identification or speaker diarization techniques, including analysis of acoustic features, voice embeddings, temporal separation, spatial audio cues, or probabilistic classification models. In such embodiments, the visual display screen may present subtitles with visual differentiation indicating distinct speakers, including labels such as “patient,” “staff,” “speaker 1”, “speaker 2”, “speaker 3”, or “other,” or visual attributes including color coding, positional grouping, or textual prefixes to indicate the source of detected speech. In some embodiments, subtitles corresponding to patient speech detected by the microphone are displayed in a first visual format, while subtitles corresponding to speech received from a remote caregiver or receiving system are displayed in a second visual format. In other embodiments, subtitles corresponding to ambient or third-party speech detected in the environment are displayed with reduced prominence or marked as background audio. This differentiation enables the patient to visually understand ongoing communication and contextual speech without relying solely on audio output.

In some embodiments, subtitle generation is performed locally by the processor of the wireless call light device. In other embodiments, captured audio is transmitted to a centralized processing hub or cloud-based system for speech recognition processing, and the resulting subtitle text is transmitted back to the device for display. In still other embodiments, subtitle generation is performed using a hybrid approach in which preliminary transcription is performed locally and refined remotely. In some embodiments, the artificial intelligence interface selectively enables subtitle display based on user preferences, accessibility settings, detected hearing impairment, environmental noise levels, or system configuration. For example, subtitle display may be automatically enabled when ambient noise exceeds a predetermined threshold or when a patient preference for visual communication is stored in memory. In some embodiments, subtitles generated from detected speech are displayed in synchronization with audio output generated by the speaker, thereby providing multimodal communication support. In other embodiments, subtitles are displayed independently of audio output, including when audio output is muted or unavailable. The foregoing embodiments provide sufficient structural and operational detail to enable generation, differentiation, and display of speech-derived textual content corresponding to multiple recognized voices without undue experimentation by a person of ordinary skill in the art.

In some embodiments, the wireless call light device or an associated receiving system is configured to support registration of authorized caregiver voices for display of caregiver-identifying information on the visual display screen. Voice registration may be performed by a staff member during an onboarding, authentication, or configuration process and is not required for operation of the device. In some embodiments, a caregiver registers a voice profile by providing one or more spoken samples while authenticated as an authorized user. Authentication may be performed using a staff credential, badge scan, proximity-based authorization, secure login, or other access control mechanism. The spoken samples are processed by the artificial intelligence interface to generate a voice representation sufficient to distinguish the caregiver's voice from other detected voices during operation. In some embodiments, the registered voice profile is associated with non-clinical identifying metadata, including but not limited to a caregiver name, role, title, or designation, such as “Nurse,” “Physician,” “Technician,” or “Respiratory Therapist.” The associated metadata may be stored locally on the device, at a centralized local processing hub, or in a secure remote system, and may be linked to the voice profile using an identifier rather than storing raw audio data. In some embodiments, when the artificial intelligence interface detects speech captured by the microphone that matches a registered caregiver voice profile above a confidence threshold, the visual display screen presents subtitle text corresponding to the detected speech together with the associated caregiver identifier. For example, the display may show “Nurse Smith:” followed by subtitle text representing spoken words detected from the caregiver.

In some embodiments, when detected speech does not match any registered caregiver voice profile with sufficient confidence, the display presents the speech using a generic speaker identifier, such as “Speaker 1,” “Speaker 2,” or similar, as previously described. This fallback ensures continuous subtitle operation without requiring voice registration. In some embodiments, the confidence threshold for displaying a caregiver name or title is configurable. When the confidence threshold is not satisfied, the artificial intelligence interface suppresses display of identifying metadata and instead displays a generic speaker identifier to reduce misattribution. In some embodiments, caregiver voice registration is limited to identifying communication participants and does not enable or perform medical diagnosis, clinical decision-making, or patient assessment. Display of caregiver identifiers is provided solely to enhance communication clarity and situational awareness for the patient and staff. In some embodiments, caregiver voice profiles may be updated, revoked, or disabled by authorized administrators. Voice profiles may be automatically invalidated upon expiration of staff credentials, role changes, or removal of authorization.

In some embodiments, the artificial intelligence interface performs voice matching and subtitle generation in real time without retaining long-term recordings of caregiver speech. In other embodiments, temporary audio buffers are used for processing and discarded after subtitle generation, in accordance with privacy or institutional policy. In some embodiments, the visual display screen presents caregiver identifiers, roles, or titles only during active communication sessions and removes such identifiers when communication ends or when no speech is detected. In some embodiments, the artificial intelligence interface is configured to assign generic speaker identifiers to detected speech that does not correspond to a registered voice profile. The generic speaker identifiers are assigned in a numerical sequence based on the order in which unrecognized voices are detected during a communication session. In some embodiments, upon detecting a first unrecognized voice, the artificial intelligence interface assigns the identifier “Speaker 1” to the detected speech and causes the visual display screen to present subtitle text prefixed with the identifier “Speaker 1.” Upon detecting a second unrecognized voice distinct from the first unrecognized voice, the artificial intelligence interface assigns the identifier “Speaker 2,” and so forth for additional unrecognized voices, including “Speaker 3,” “Speaker 4,” and additional numerical identifiers as needed.

In some embodiments, the artificial intelligence interface distinguishes between different unrecognized voices using acoustic characteristics of the captured audio, including one or more of voice timbre, pitch range, speech cadence, spectral features, or other voice separation techniques. The artificial intelligence interface is configured to maintain consistency of assigned numerical identifiers for each unrecognized voice throughout an active communication session, such that repeated speech from the same unrecognized voice is labeled with the same numerical identifier. In some embodiments, when an unrecognized voice ceases to be detected for a predetermined duration, the artificial intelligence interface may retire the corresponding numerical identifier. In such embodiments, newly detected unrecognized voices may be assigned the next available numerical identifier or may reuse retired identifiers, depending on system configuration. In some embodiments, numerical speaker identifiers are reset upon termination of the communication session, clearing of the latched call state, expiration of a session timer, or receipt of an authorized reset command. This ensures that numerical identifiers are not persistently associated with individuals across sessions. In some embodiments, the artificial intelligence interface performs numerical speaker labeling without storing identifying information about the unrecognized voices and without associating the numerical identifiers with patient identity, caregiver identity, or medical information. The numerical identifiers are used solely for communication clarity and subtitle differentiation.

In some embodiments, subtitle text associated with numerical speaker identifiers is generated in real time based on audio captured by the microphone and is displayed on the visual display screen concurrently with detected speech. The subtitle text may represent recognized words, partial words, or phonetic approximations when speech clarity is limited. In some embodiments, if voice separation confidence falls below a predetermined threshold, the artificial intelligence interface may temporarily suspend numerical differentiation and display speech using a single generic identifier until sufficient confidence is restored. This fallback behavior ensures reliable operation without mislabeling. In some embodiments, numerical speaker labeling operates independently of caregiver voice registration, such that the device continues to provide subtitle output and speaker differentiation even when no registered voice profiles are present or enabled.

### Determination of Confidence Level by the Artificial Intelligence Interface

In some embodiments, the artificial intelligence interface is configured to determine a confidence level associated with interpretation of patient input prior to initiating one or more device actions. The confidence level represents a quantitative or qualitative measure of the reliability, certainty, or accuracy of the artificial intelligence interface in interpreting the patient input.

The patient input may include, but is not limited to, spoken audio captured by a microphone, non-verbal vocalizations, text input, touchscreen input, button selections, gesture input, or combinations thereof. The confidence level may be determined using one or more artificial intelligence models, algorithms, or decision frameworks executed by the processor.

In some embodiments, the confidence level is calculated as a numerical score expressed as a percentage, probability value, normalized score, or confidence metric derived from the output of one or more artificial intelligence models. The confidence level may be calculated using one or more non-limiting techniques, either individually or in combination.

In some embodiments, the artificial intelligence interface comprises a classification model, neural network, or large language model that generates one or more candidate interpretations of patient input. The confidence level may be determined based on a probability value assigned by the model to a selected interpretation relative to alternative interpretations. For example, a confidence level of approximately eighty percent may indicate that the model assigns an approximately 0.80 probability to a particular intent classification or action determination.

In some embodiments, the confidence level is derived from normalized output values, including softmax probabilities, confidence logits, attention-weight distributions, or other normalized scores produced by a machine learning model. These values may be converted into percentage-based confidence levels by scaling or normalization relative to a maximum possible output score.

In some embodiments, the artificial intelligence interface comprises multiple models, sub-models, or inference passes. The confidence level may be determined based on agreement among outputs generated by the multiple models or inference passes. When multiple outputs independently produce the same interpretation or recommended device action, the confidence level may be increased. Conversely, disagreement among outputs may reduce the confidence level.

In embodiments involving speech recognition or natural language processing, the confidence level may be based on transcription accuracy scores, word error rates, intent classification certainty, semantic similarity scores, contextual coherence measures, or combinations thereof generated by the artificial intelligence interface.

In some embodiments, the confidence level is adjusted based on signal quality metrics associated with the patient input, including background noise level, signal-to-noise ratio, speech clarity, microphone quality, detected speech artifacts, or combinations thereof. Reduced signal quality may result in a reduced confidence level even when an interpretation is generated.

In some embodiments, the confidence level is influenced by consistency between the interpreted patient input and contextual information, including patient history, prior device interactions, device state, time of day, hospital workflow context, or combinations thereof. Interpretations that are inconsistent with contextual information may result in a reduced confidence level.

In some embodiments, the device compares the determined confidence level to one or more predetermined threshold values to determine whether automated interpretation is sufficiently reliable. In some embodiments, the predetermined threshold is approximately eighty percent, such that confidence levels at or below this threshold cause the device to enter a fallback mode. In other embodiments, the predetermined threshold is approximately seventy percent, approximately sixty percent, or approximately fifty percent.

In some embodiments, the threshold value is adjustable by hospital administrators, healthcare providers, regulatory settings, patient safety policies, or device configuration profiles.

In some embodiments, the confidence threshold is dynamically adjusted based on operating conditions. For example, higher thresholds may be applied in emergency or high-risk scenarios, while lower thresholds may be permitted for routine or non-critical requests. In some embodiments, the threshold may be dynamically modified based on patient condition, patient communication ability, prior successful interactions, device learning history, system performance metrics, or combinations thereof.

In some embodiments, when the confidence level fails to satisfy the predetermined threshold, the artificial intelligence interface initiates a fallback mode. In fallback mode, the device may generate a generic assistance request, transmit an indication that interpretation confidence is limited, and transmit raw or minimally processed patient input, including recorded audio input or text-based input, to a receiving system for human review.

The foregoing embodiments describe multiple concrete techniques by which confidence levels may be calculated, quantified, and evaluated by the artificial intelligence interface. These techniques provide sufficient structure and operational detail to enable a person of ordinary skill in the art to implement the confidence determination and threshold-based control logic without undue experimentation.

In one exemplary embodiment, a confidence level is calculated by comparing a probability score assigned to a selected interpretation of patient input to a sum of probability scores assigned to alternative interpretations. For example, when a speech recognition and intent classification model assigns a probability of approximately 0.78 to a first interpretation and lower probabilities to remaining interpretations, the confidence level may be determined as approximately seventy-eight percent. If the confidence level is below a predetermined threshold, the device initiates the fallback mode. This example is provided for illustration only, and other confidence calculation techniques may be used.

### Recorded Audio Capture and Transmission in Fallback Mode

In some embodiments, the wireless call light device is configured to capture and record patient audio input received via the microphone during operation of the artificial intelligence interface. The recorded audio input may be captured continuously, intermittently, or in response to detection of patient speech or non-verbal vocalizations.

In some embodiments, the recorded audio input is temporarily stored in memory while the artificial intelligence interface processes the patient input and determines a confidence level associated with interpretation of the input. The recorded audio may be buffered for a predetermined duration, including several seconds before and after detection of patient speech, to preserve contextual information.

In some embodiments, when the determined confidence level satisfies the predetermined threshold, the recorded audio input is automatically erased, overwritten, or discarded from memory without transmission to a receiving system. This embodiment reduces unnecessary data transmission and protects patient privacy.

In some embodiments, when the confidence level fails to satisfy the predetermined threshold, the device enters a fallback mode in which the recorded audio input is transmitted directly to a receiving station, nursing station, central communication system, or other hospital system. The transmitted audio may allow healthcare personnel to directly review the patient's original input without reliance on automated interpretation.

In some embodiments, the recorded audio input is transmitted in association with an assistance request and an indication that interpretation confidence is limited, thereby alerting receiving personnel that the transmitted audio should be reviewed manually.

In some embodiments, the recorded audio input may be transmitted in real time, near real time, or as a stored audio clip, depending on system configuration, network availability, or hospital policy. The audio may be transmitted using wired or wireless communication protocols, including Wi-Fi, radio frequency, cellular, or hospital network infrastructure.

In some embodiments, the device may transmit both the recorded audio input and a corresponding text-based transcription generated by the artificial intelligence interface, thereby allowing receiving personnel to compare automated transcription results with the original audio.

In some embodiments, storage and transmission of recorded audio input may be controlled by configurable privacy settings, retention policies, or regulatory compliance requirements. For example, recorded audio may be automatically deleted after transmission, retained for a limited duration, or stored in a secure database associated with patient care records.

The foregoing embodiments provide sufficient structural and operational detail to enable capture, temporary storage, conditional transmission, and disposal of recorded patient audio input in connection with confidence-based fallback operation of the artificial intelligence interface, without undue experimentation by a person of ordinary skill in the art.

In some embodiments, the wireless call light device is configured to transmit assistance requests as coded event signals determined based on patient input. The coded event signals represent discrete event types rather than diagnostic or clinical data and are generated to communicate the nature of a patient request in a standardized, machine-readable form.

In some embodiments, patient input may include activation of a patient call button, input received through a microphone, interaction with a display screen, haptic input, or input interpreted by an artificial intelligence interface executed by the processor. Based on the patient input, the device determines an appropriate event type to be transmitted as a coded event signal.

In some embodiments, the coded event signals represent one or more event types including an assistance request event, a communication request event, or a general alert event. An assistance request event may indicate that a patient requires in-person assistance. A communication request event may indicate that a patient is requesting two-way communication with hospital staff. A general alert event may indicate a non-specific or informational condition requiring staff awareness without conveying diagnostic information.

In some embodiments, the coded event signals are generated by the processor in response to patient input and transmitted via the communication portal to a receiving system, central communication system, or hospital information system. The coded event signals may include event identifiers, priority indicators, timestamps, or device identifiers, and may be transmitted using wired or wireless communication protocols, including Wi-Fi, radio frequency, cellular, or hospital network infrastructure.

In some embodiments, the use of coded event signals allows the system to communicate patient needs without transmitting diagnostic data, medical assessments, or clinical determinations. This event-based signaling architecture enables efficient routing, prioritization, logging, and response handling by receiving systems while reducing data complexity and preserving patient privacy.

In some embodiments, the coded event signals may be generated directly from patient input or indirectly through processing performed by the artificial intelligence interface. For example, the artificial intelligence interface may interpret patient input and select an appropriate event type based on predefined rules, learned models, or confidence thresholds. In other embodiments, the event type may be selected without use of artificial intelligence based solely on detected patient interaction.

In some embodiments, the event-based signaling framework allows receiving systems to process assistance requests uniformly across multiple devices, locations, or hospital departments, regardless of the specific form of patient input that initiated the event. The foregoing embodiments enable transmission of assistance requests as coded event signals determined based on patient input.

In some embodiments, the artificial intelligence processing module is configured to process captured voice signals received from the microphone of the wireless call light device. The captured voice signals may include spoken words, partial words, unintelligible speech, non-word vocalizations, non-verbal utterances, or combinations thereof, as well as ambient noise associated with patient interaction. Prior to interpretation, the artificial intelligence processing module may perform one or more preprocessing operations including, but not limited to, noise reduction, signal normalization, segmentation into audio frames, filtering, and voice activity detection to isolate patient-generated audio signals.

In some embodiments, the artificial intelligence processing module is configured to interpret patient intent or assistance requests based on analysis of the captured voice signals. Interpretation of patient intent may be performed using speech recognition models, natural language processing models, intent classification models, probabilistic classifiers, rule-based logic, or combinations thereof. In certain embodiments, the artificial intelligence processing module determines whether the patient is requesting physical assistance, communication with staff, or general attention based on recognized words, partial linguistic content, temporal audio patterns, repetition, or contextual device state.

In some embodiments, the artificial intelligence processing module is further configured to recognize impaired, unclear, or non-standard speech patterns, as well as non-word vocalizations. Impaired speech patterns may include speech affected by medical conditions, neurological impairment, respiratory distress, pain, fatigue, emotional distress, or reduced motor control. Unclear speech patterns may include slurred speech, whispered speech, fragmented speech, low-amplitude speech, or speech obscured by environmental noise. Non-standard speech patterns may include atypical pronunciation, incomplete words, repeated syllables, vocal strain, or irregular articulation. Non-word vocalizations may include sounds lacking linguistic structure, including groans, moans, gasps, cries, repeated phonetic noises, or other vocal emissions not intended to form words.

In some embodiments, recognition of impaired, unclear, non-standard speech patterns, or non-word vocalizations is performed by analyzing acoustic features of the captured voice signals. Such features may include pitch variability, amplitude envelope, temporal duration, spectral content, articulation timing, pause frequency, repetition rate, or signal-to-noise ratio. In other embodiments, recognition is performed by evaluating confidence metrics produced by speech recognition or intent classification models, including recognition confidence scores, probability distributions, entropy values, or likelihood rankings associated with candidate interpretations. In further embodiments, recognition may be performed by detecting an absence of linguistic structure, failure to form recognizable lexical units, or repeated detection of vocal energy without corresponding word-level hypotheses.

In some embodiments, the artificial intelligence processing module distinguishes non-word vocalizations from uncertain word recognition by determining that the captured voice signals do not correspond to identifiable words or phrases within a language model, rather than merely exhibiting ambiguity between multiple possible word interpretations. In such embodiments, non-word vocalizations are identified as patient input conveying distress or attention-seeking behavior without linguistic intent.

In some embodiments, upon interpretation of patient input, including detection of impaired, unclear, non-standard speech patterns, or non-word vocalizations, the artificial intelligence processing module converts the patient input into standardized assistance events. Standardized assistance events comprise structured, non-diagnostic representations of patient requests suitable for transmission via the communication portal. The standardized assistance events may be represented as coded event signals, event identifiers, or data packets independent of raw audio content.

In some embodiments, standardized assistance events include one or more of an assistance request event indicating a need for in-person help, a communication request event indicating a request for two-way interaction, or a general alert event indicating a non-specific need for staff awareness. In further embodiments, standardized assistance events may include associated metadata including priority indicators, urgency levels, timestamps, device identifiers, or patient location identifiers.

In some embodiments, data transmitted to a receiving system in connection with a standardized assistance event includes the event type, device identifier, assigned bed identifier, time of event generation, and optionally an indication of interpretation confidence. In certain embodiments, diagnostic conclusions, medical assessments, or clinical determinations are expressly excluded from the transmitted data.

In some embodiments, when impaired, unclear, non-standard speech patterns or non-word vocalizations are detected, the artificial intelligence processing module may additionally transmit supplemental information, including recorded audio segments, partial text representations, or indicators of interpretation uncertainty. In other embodiments, recorded audio is stored locally and transmitted only when a confidence threshold is not satisfied or when explicitly requested by the receiving system.

In some embodiments, the conversion of patient input into standardized assistance events enables the receiving system to respond efficiently without requiring interpretation of raw audio signals, thereby facilitating prioritization, preserving patient privacy, and reducing cognitive load on receiving personnel.

### Microcontroller/System-on-Chip Enablement Statement

In some embodiments, the wireless call light device includes a controller implemented as a microcontroller, a system on chip (SoC), or a combination of processing components integrated within a single package or distributed across multiple packages. The controller is operatively coupled to one or more hardware and software components of the device and is configured to coordinate operation of the device in accordance with the functions described herein.

In various embodiments, the controller is operatively coupled to memory storing executable instructions, configuration data, identifiers, artificial intelligence models, thresholds, and event definitions. The controller executes stored instructions to perform device functions including, but not limited to, receiving patient input, controlling communication operations, managing indicator light states, executing artificial intelligence logic, generating coded event signals, and coordinating fallback behavior when interpretation confidence thresholds are not satisfied.

In some embodiments, the controller is operatively coupled to the microphone and speaker through one or more audio interfaces, codecs, amplifiers, or digital signal processing components. The controller is configured to receive captured audio signals from the microphone, process or route the audio signals for artificial intelligence interpretation, record audio segments in memory, and output audio prompts, confirmations, or two-way communication audio through the speaker.

In some embodiments, the controller is operatively coupled to one or more patient input devices, including the patient call button, flexible cord button, staff-only reset button or second user reset button, emergency buttons, touch inputs, or combinations thereof. The controller detects actuation events, debounces button inputs, enforces resistive or deliberate-press mechanisms, and determines appropriate device actions based on the detected input and current device state.

In some embodiments, the controller is operatively coupled to one or more output devices, including indicator lights, display screens, haptic output devices, and audio output components. The controller selectively activates, deactivates, or modulates these outputs to provide visual, tactile, and audio feedback to the patient or staff, including persistent illumination, flashing patterns, vibration patterns, displayed text or graphics, and audio confirmations.

In some embodiments, the controller is operatively coupled to a wireless communication module configured to transmit and receive data using one or more wireless protocols. The controller formats and transmits coded event signals, assistance requests, audio data, text data, identifiers, and status information to a receiving system, and may implement acknowledgment handling, retry logic, encryption, or buffering as required.

In some embodiments, the artificial intelligence interface is executed by the controller itself or by a dedicated processing core within the microcontroller or system on chip. In other embodiments, the artificial intelligence interface is distributed between on-device processing and remote processing resources, with the controller coordinating data exchange, result handling, and device actions based on artificial intelligence output.

In some embodiments, the controller manages power delivery and power states of the device by interfacing with power management circuitry, battery monitoring circuits, and charging interfaces. The controller may monitor battery status, generate low-battery alerts, and support hot-swappable battery operation without interruption of device functionality.

In some embodiments, the controller coordinates multiple components concurrently, allowing the device to receive patient input, execute artificial intelligence interpretation, generate standardized assistance events, activate multimodal outputs, and communicate with a receiving system without requiring manual intervention by staff.

The foregoing embodiments describe representative implementations in which a microcontroller or system on chip is operatively coupled to device components to perform the functions described herein. Other configurations, component arrangements, and processing architectures may be used without departing from the scope of the invention.

### Central Hub-Based Artificial Intelligence Processing (Embodiment)

In some embodiments, the artificial intelligence processing module is executed by a centralized on-premises hub or local processing node that is communicatively coupled to one or more wireless call light devices. The central hub may be located within a healthcare facility, department, unit, or designated local network environment and is distinct from cloud-based systems accessible over a public network.

In such embodiments, patient input captured by a wireless call light device, including voice signals, non-verbal vocalizations, or physical input events, is transmitted to the central hub for artificial intelligence processing. The central hub executes one or more artificial intelligence models configured to interpret patient intent, recognize impaired, unclear, or non-standard speech patterns or non-word vocalizations, and generate standardized assistance events based on the interpreted input.

In some embodiments, the central hub returns the standardized assistance event to the originating wireless call light device for subsequent transmission to a receiving system. In other embodiments, the central hub directly transmits the standardized assistance event to the receiving system on behalf of the wireless call light device. In still other embodiments, the central hub coordinates processing and routing of assistance events for multiple wireless call light devices simultaneously.

In some embodiments, the central hub performs confidence level determination associated with interpretation of patient input and applies fallback logic when confidence thresholds are not satisfied, including generation of generic assistance requests and transmission of indications that interpretation confidence is limited. In certain embodiments, recorded audio or text-based input associated with the patient input is temporarily buffered at the central hub and retained, discarded, or transmitted in accordance with confidence thresholds, acknowledgment signals, or system configuration settings.

In some embodiments, execution of artificial intelligence processing at the central hub reduces computational requirements on individual wireless call light devices while maintaining local network control, reduced latency, enhanced privacy, and independence from external cloud infrastructure. The central hub may comprise one or more processors, memory resources, and communication interfaces and may operate as part of a hub-and-node architecture, mesh architecture, or hybrid topology.

In some embodiments, the wireless call light devices are selectively configurable to utilize on-device artificial intelligence processing, central hub-based artificial intelligence processing, cloud-based artificial intelligence processing, or any combination thereof, based on deployment requirements, network availability, or system policies.

In some embodiments, the wireless call light device includes a processing device, such as a microcontroller, system-on-a-chip, application-specific integrated circuit, or other integrated processing circuit, configured to operatively couple and coordinate all functional components of the device. The processing device is configured to execute stored instructions to receive and process patient input, manage communication operations, control audio, visual, and haptic output devices, store and retrieve identifiers and configuration data from memory, execute artificial intelligence logic, generate standardized assistance events, monitor confidence thresholds, enforce latched call states, manage acknowledgment and retry logic, and control power management functions.

In some embodiments, the processing device is further configured to interface with communication circuitry to transmit and receive data with one or more receiving systems, central hubs, or cloud-based platforms, and to route patient input and artificial intelligence outputs between local and remote processing resources. In certain embodiments, the processing device selectively enables on-device processing, central hub-based processing, cloud-based processing, or hybrid processing modes based on system configuration, network availability, or operational policy, while maintaining continuous device functionality.

In some embodiments, the processing device synchronizes operation of multiple hardware and software components concurrently, such that patient input, artificial intelligence interpretation, event generation, output activation, and communication transmission occur without requiring manual intervention by healthcare staff. The processing device thereby serves as a centralized control element that integrates all device subsystems into a coordinated wireless call light platform.

### Continuous Audio Capture and Retrospective Transmission Embodiment

In some embodiments, the wireless call light device is configured to continuously capture audio using the microphone during normal operation of the device. The captured audio is temporarily buffered or stored in memory as a rolling audio record, such that older audio data is automatically overwritten as new audio data is captured. The rolling audio record may comprise a predetermined duration of captured audio, including but not limited to up to five minutes of audio preceding a given point in time.

In some embodiments, the captured audio is stored locally in memory of the wireless call light device. In other embodiments, the captured audio is transmitted in real time or near real time to a centralized local processing hub, a cloud-based storage system, or a combination thereof for storage. In further embodiments, the wireless call light device selectively stores portions of the captured audio locally while transmitting other portions to a remote storage system, based on available bandwidth, system configuration, privacy settings, or operational policy.

In some embodiments, upon receipt of a user input by the wireless call light device, including activation of a physical call initiation control or receipt of patient input processed by the artificial intelligence interface, the processing device retrieves a portion of the stored audio corresponding to a time interval preceding the user input and a time interval following the user input. The retrieved audio may include up to approximately five minutes of audio recorded prior to receipt of the user input and up to approximately five minutes of audio recorded after receipt of the user input.

In some embodiments, the retrieved audio is transmitted to a receiving system together with an assistance request, coded event signal, or other notification generated by the wireless call light device. The transmitted audio may be used by receiving personnel to review patient context, assess urgency, or supplement automated interpretation of patient input. In certain embodiments, transmission of the retrieved audio occurs automatically in response to the user input, without requiring additional manual action by healthcare staff.

In some embodiments, storage and transmission of continuously captured audio is controlled by one or more privacy, retention, or compliance settings. For example, audio data may be encrypted during storage and transmission, retained only for a limited duration, automatically deleted after acknowledgment of receipt by the receiving system, or selectively transmitted based on confidence thresholds associated with interpretation of patient input. In certain embodiments, continuously captured audio that is not transmitted is automatically discarded or overwritten to minimize data retention.

In some embodiments, continuous audio capture and retrospective transmission operate independently of whether artificial intelligence interpretation is performed locally on the wireless call light device, via a centralized processing hub, or via a cloud-based system. The processing device coordinates buffering, retrieval, and transmission of audio data while maintaining uninterrupted operation of other device functions, including call initiation, indicator light control, two-way communication, and power management.

In some embodiments, an authorized reset mechanism refers to a hardware, software, or combined control feature that terminates a latched call state, re-enables processing of patient call initiation inputs, and/or extinguishes or changes an indicator light state only after a reset action is performed by an authorized party, including clinical staff, caregivers, administrators, or an authorized system. The authorized reset mechanism is configured such that a patient or other unauthorized user is prevented from resetting the device, thereby reducing nuisance resets, preserving event integrity, and ensuring that a call request remains active until appropriately addressed. In some embodiments, the authorized reset mechanism requires authentication, authorization, or access control before a reset command is accepted and executed by the wireless call light device. Authentication may be performed locally at the device, remotely via a receiving system, or using a hybrid approach, and may be implemented using credential validation, cryptographic verification, role-based access control, proximity verification, physical access restriction, or combinations thereof.

In some embodiments, the authorized reset mechanism includes a staff-authenticated action that must be successfully completed before the device exits the latched call state and clears an active call indication. In such embodiments, the device is configured to accept a reset command only upon receiving staff authentication data indicating that the reset action is being performed by an authorized caregiver. In certain embodiments, the staff-authenticated action includes a badge tap or badge scan, wherein the device includes or is coupled to an RFID or NFC reader configured to read a staff badge credential. Upon detection of a valid credential, the processor validates the credential locally against stored authorized identifiers or remotely via a hospital authentication server and enables the reset operation. In other embodiments, the staff-authenticated action includes a secure login or credential entry, wherein the device prompts for staff credentials including a personal identification number, password, biometric input, or secure mobile application authentication, and executes the reset only after successful credential verification. In further embodiments, the staff-authenticated action includes authorized device proximity, wherein the device determines whether an authorized staff device is within a predetermined proximity threshold using Bluetooth Low Energy, ultra-wideband, Wi-Fi association, cryptographic handshake, or similar techniques, and enables the reset operation only when the proximity condition and authorization requirement are satisfied. In some embodiments, execution of a staff-authenticated reset causes the device to generate and store a reset record in memory or transmit a reset confirmation to a receiving system, including a timestamp and optional staff identifier, to support auditing and workflow tracking.

In some embodiments, the authorized reset mechanism includes a remote reset command issued by an authorized caregiver console, nurse station workstation, centralized processing hub, or other receiving system. In such embodiments, the wireless call light device is configured to receive a reset message via the communication portal and to execute the reset operation only when the reset message satisfies one or more authorization requirements. Authorization requirements may include verification that the reset command originates from a whitelisted system address, device identifier, or authenticated console account, validation of a cryptographic signature, token, or certificate included with the reset command, confirmation that the reset command is associated with a correct device identifier, patient support apparatus identifier, or active event identifier, and role-based permission checks indicating that the sender has authorization to reset calls for the applicable device, unit, or patient location. In some embodiments, upon receipt of an authorized remote reset command, the processor transitions the device from the latched call state to a non-latched state, updates the indicator light state, and transmits an acknowledgment message back to the authorized console or hub confirming completion of the reset.

In some embodiments, the authorized reset mechanism includes a physical reset interface that is inaccessible to the patient and reserved for staff use. In such embodiments, the physical reset interface comprises a staff-only control feature that is positioned, shielded, locked, or otherwise configured to prevent patient access during ordinary use. In certain embodiments, the physical reset interface includes a recessed or covered reset button requiring a tool, key, or specialized actuator, a reset switch located on a portion of the device or mounting assembly oriented away from patient reach when the device is attached to a bed rail or IV pole, a reset control disposed within a lockable housing or behind a tamper-resistant panel, or a reset interface that requires opening a staff-accessible compartment to physically access the reset control. In some embodiments, activation of the staff-only physical reset interface causes the processor to clear the active call state, terminate the latched call state, and extinguish or modify the indicator light state. In certain embodiments, the device records occurrence of the reset in memory, including a timestamp or reset counter, for auditability.

In some embodiments, the authorized reset mechanism is implemented using any combination of staff-authenticated actions, remote reset commands, and staff-only physical reset interfaces. For example, the device may require both a staff authentication event and receipt of a remote reset command before clearing the latched call state. In other embodiments, when network connectivity is unavailable, the device permits reset via the staff-only physical reset interface while disallowing reset via patient-accessible controls. In some embodiments, the authorized reset mechanism is configured to reject reset commands that fail authentication, originate from unknown sources, do not satisfy proximity requirements, or are attempted using patient-accessible interfaces, thereby ensuring that reset operations are performed only by authorized personnel or systems.

As used herein, a ‘call light device’ (also referred to as a ‘nurse call device,’ ‘patient call device,’ or ‘assistance request device’) refers to any patient-accessible electronic device configured to generate and transmit a patient-initiated request, alert, or communication to a receiving system or caregiver, whether by wired or wireless communication. A call light device may be portable, wearable, handheld, bed-mounted, pole-mounted, rail-mounted, or otherwise associated with a patient support apparatus, and may include one or more input interfaces (e.g., button, corded control, voice interface), output interfaces (e.g., indicator light, speaker, display, haptic output), and communication circuitry for transmitting an event, message, or call request.

In some embodiments, the wireless call light device and associated systems are configured solely to facilitate communication, coordination, and routing of patient-initiated inputs, alerts, or assistance requests. The system does not determine, diagnose, confirm, or assess medical conditions and does not provide clinical decision support, diagnostic output, or treatment recommendations.

In such embodiments, any artificial intelligence functionality described herein is limited to interpretation, classification, formatting, or transmission of patient inputs for presentation to human caregivers or receiving systems. The system does not autonomously make medical determinations, clinical judgments, or care decisions. Rather, all medical interpretation, prioritization, and response actions remain under the control and discretion of qualified healthcare personnel.

Accordingly, the system operates as a communication and coordination platform that assists in conveying patient-initiated information to human reviewers, without replacing or supplanting clinical evaluation, diagnosis, or treatment by healthcare professionals.

In some embodiments, “patient-facing software functions” refers to software-implemented functions of the wireless call light device that are configured to directly interact with, receive input from, or present output to a patient during operation of the device. Patient-facing software functions may include, but are not limited to, software routines that manage patient input interfaces, present audio, visual, or haptic output to the patient, provide prompts, confirmations, or status indications, process patient voice input, display messages or indicators on a display screen, or otherwise facilitate patient interaction with the device. Patient-facing software functions may be executed by the processor alone or in cooperation with the artificial intelligence interface and may control or coordinate operation of one or more hardware components of the device.

In some embodiments, a “hot-swappable battery” refers to a power source that may be removed from and replaced in the wireless call light device without interrupting operation of the device, including without loss of power to critical components, without device shutdown, and without loss of active call state, data, or communication functionality. In such embodiments, temporary power continuity may be maintained using one or more auxiliary energy storage elements, internal reserve batteries, capacitors, supercapacitors, power management circuits, or combinations thereof.

As used herein, a “patient support apparatus” includes any structure configured to support, transport, or position a patient, including hospital beds, stretchers, gurneys, wheelchairs, transport carts, procedure tables, or similar devices.

As described herein, the wireless call light device is configured to generate, transmit, and manage multiple categories of patient-initiated or staff-initiated alerts, each corresponding to a predefined alert type supported by the device and the associated receiving system. Such alert types include, but are not limited to: assistance request alerts, communication request alerts, general alerts, urgent alerts, rapid response alerts, and code-related emergency alerts, including Code Blue alerts.

Each alert type may be initiated via physical input controls, including a patient call button, flexible cord control, staff-accessible emergency buttons, or other authorized input interfaces, and/or via voice-based input interpreted by an artificial intelligence interface, wherein verbal commands are mapped to the same predefined alert types as the corresponding physical controls. In voice-based embodiments, the artificial intelligence interface is configured to receive audio input captured by a microphone and to process the audio input using one or more speech recognition, keyword detection, pattern-matching, probabilistic classification, or machine-learning models executed by a processor. The artificial intelligence interface analyzes the captured audio to identify predefined trigger phrases, keywords, vocal patterns, or non-verbal vocalizations associated with existing alert categories supported by the device. Based on the analysis, the artificial intelligence interface selects one of the predefined alert types corresponding to the interpreted patient intent.

The artificial intelligence interface does not generate new alert categories and does not perform medical diagnosis, clinical assessment, or clinical decision support. Instead, the artificial intelligence interface functions as an alternative input mechanism that translates voice input into the same standardized assistance events or coded event signals that would be generated by activation of the corresponding physical input controls.

In certain embodiments, the artificial intelligence interface determines a confidence level associated with interpretation of the voice input. When the confidence level satisfies a predetermined threshold, the selected alert type is initiated in the same manner as if initiated by a physical control. When the confidence level fails to satisfy the predetermined threshold, the device enters a fallback mode in which a generic assistance request is generated and supplemental information, including recorded audio or text-based representations of the voice input, is transmitted to a receiving system for human review.

What is claimed is:

1. A wireless call light device comprising:
a communication portal configured to place a call to a pre-programmed recipient upon activation of a call initiation control;
a speaker and microphone for enabling two-way audio communication between a first user and a second user;
a memory and processor configured to store a user-programmable identifier corresponding to a patient support apparatus and transmit the assigned identifier to a central database system when a call is placed;
an indicator light configured to Illuminate when a call is initiated;
configured to remain illuminated until manually turned off via an authorized reset mechanism;
and configured to enter a latched call state that suppresses processing of repeated call initiation inputs until receipt of an authorized reset command;
a call initiation control mounted on the enclosure, or a remotely coupled call initiation control comprising a flexible cord and a grip portion;
a power source, including a hot-swappable battery housed within a battery compartment;
a holding clip or a rod attachment mechanism configured to secure the device to a patient support apparatus or IV pole; and
an artificial intelligence interface executed by the processor and operatively coupled to patient-facing software functions and controllable hardware components of the device, the artificial intelligence interface being configured to receive patient input and determine one or more device actions to be performed.

2. The wireless call light device of claim 1, further comprising a haptic output device operatively coupled to the wireless call light device, wherein the haptic output device is configured to provide tactile feedback to a patient, and wherein the haptic output device is coupled to the wireless call light device by a wired connection, a wireless connection, or a combination thereof.

3. The wireless call light device of claim 1, further comprising a display screen, wherein the artificial intelligence interface is configured to receive input from the microphone and provide output via the speaker and the display screen.

4. The wireless call light device of claim 1, wherein the artificial intelligence interface is further configured to determine a confidence level associated with interpretation of patient input, and wherein, when the confidence level fails to satisfy a predetermined threshold, the device is configured to enter a fallback mode that generates an assistance request, transmits an indication to a receiving system that interpretation confidence is limited, and further transmits at least one of recorded audio input or text-based input associated with the patient input directly to the receiving system.

5. The wireless call light device of claim 1, 2, or 3, further comprising multimodal output to a patient, the multimodal output comprising one or more of haptic output, visual output, and audio output, generated in response to patient input, device state, system state, or operation of the artificial intelligence interface.

6. The wireless call light device of claim 1, wherein the device is configured to transmit assistance requests as coded event signals determined based on patient input, the coded event signals representing one or more event types selected from an assistance request event, a communication request event, or a general alert event.

7. The wireless call light device of claim 1, wherein the artificial intelligence interface comprises an artificial intelligence processing module configured to operate locally on the device, remotely via a cloud-based system, or a combination thereof, and wherein the artificial intelligence processing module is configured to process captured voice signals, interpret patient intent or assistance requests, recognize impaired, unclear, or non-standard speech patterns or non-word vocalizations, and convert the patient input into standardized assistance events for transmission to a receiving system.

8. The wireless call light device of claim 4, wherein the predetermined confidence threshold is selectable and includes a threshold of 80 percent or less.

9. The wireless call light device of claim 1, wherein the wireless call light device is configured to communicatively couple with one or more other wireless call light devices or one or more receiving systems, and to transmit, receive, or relay information between the wireless call light device and the one or more other wireless call light devices or the one or more receiving systems.

10. The wireless call light device of claim 7, wherein interpretation of patient input is performed locally on the wireless call light device, and wherein transmission to a remote system occurs only after generation of a standardized assistance event.

11. The wireless call light device of claim 4, wherein recorded audio associated with patient input is automatically discarded when the confidence level satisfies the predetermined threshold and is retained or transmitted only when the confidence level fails to satisfy the predetermined threshold.

12. The wireless call light device of claim 1, wherein patient input is receivable via at least one physical input interface or via the artificial intelligence interface, and wherein either input is sufficient to initiate transmission of a standardized assistance event.

13. The wireless call light device of claim 10, wherein the wireless call light device is configured to relay standardized assistance events generated by another wireless call light device when a direct communication path to the receiving system is unavailable.

14. The wireless call light device of claim 6, wherein transmission of the coded event signals includes acknowledgment monitoring and automatic retry logic when an acknowledgment is not received within a predetermined time interval.

15. The wireless call light device of claim 4, wherein recorded audio associated with patient input is retained until an acknowledgment is received from the receiving system confirming receipt of the assistance request, and wherein the recorded audio is automatically discarded by the wireless call light device after receipt of the acknowledgment.

16. The wireless call light device of claim 1, wherein the artificial intelligence interface is configured to selectively operate on the wireless call light device, via a centralized local processing hub, via a cloud-based system, or any combination thereof, to interpret patient input and generate standardized assistance events.

17. The wireless call light device of claim 1, wherein the wireless call light device is configured to continuously record audio captured by the microphone and to store the recorded audio locally on the device, at a centralized local processing hub, in a cloud-based system, or in any combination thereof, and wherein, in response to receipt of a user input by the wireless call light device, the device is configured to transmit to a receiving system a portion of the recorded audio comprising up to five minutes of audio recorded prior to receipt of the user input and up to five minutes of audio recorded after receipt of the user input.

18. A method of providing patient-initiated communication in a hospital hallway environment lacking a fixed nurse call system, the method comprising:
positioning a patient on a patient support apparatus located in a hallway or other non-room area of a healthcare facility;
mechanically coupling a wireless call light device to the patient support apparatus, the wireless call light device comprising a processor, memory, a communication portal, a call initiation control, a speaker, a microphone, an indicator light, and a power source;
assigning, by the processor, a device identifier corresponding to the hallway patient support apparatus and storing the device identifier in the memory;
receiving patient input at the wireless call light device via at least one of actuation of the call initiation control or voice input captured by the microphone;
processing the patient input using the processor to generate a standardized assistance event corresponding to the patient input;
transmitting, via the communication portal, the standardized assistance event and the device identifier to a receiving system remote from the hallway location;
illuminating the indicator light on the wireless call light device in response to generation of the standardized assistance event and maintaining the indicator light in an illuminated state until receipt of an authorized reset command;
placing the wireless call light device into a latched call state that suppresses subsequent patient input until the authorized reset command is received;
enabling two-way audio communication between the patient and hospital personnel via the speaker and the microphone; and
terminating the latched call state and extinguishing the indicator light in response to receipt of the authorized reset command after the assistance request has been addressed.
